(19) United States
(12) Patent Application Publication
(10) Pub. No.: US 2019/0167601 A1
(43) Pub. Date: Jun. 6, 2019

(54) APPARATUS AND METHOD FOR ZERO ORDER DRUG DELIVERY FROM MULTILAYER AMPHIPHILIC CO-NETWORKS

(71) Applicant: The University of Akron, Akron, OH (US)

(72) Inventors: Mukerrem CAKMAK, Lafayette, IN (US); Gustavo Andres GUZMAN CARDOZO, Pittsburgh, PA (US); Faruk ORGE, Solon, OH (US); Turgut NUGAY, Istanbul (TR)

(73) Assignee: The University of Akron, Akron, OH (US)

(21) Appl. No.: 16/313,685

(22) PCT Filed: Apr. 24, 2017
(86) PCT No.: PCT/US2017/029074

§ 371 Date: Dec. 27, 2018

Related U.S. Application Data
(60) Provisional application No. 62/356,183, filed on Jun. 29, 2016.

(51) Int. Cl. A61K 9/70; A61F 13/02; G02B 1/04; A61K 9/51

(52) U.S. Cl. CPC A61K 9/7007; A61F 13/02; A61K 45/06; A61K 9/51; G02B 1/043

(57) ABSTRACT

In one or more embodiments the present invention provides a three layer bimodal amphiphilic co-network (β-APCN) based drug delivery device and methods for its making and use. In various embodiments, the system is based on a three-layer scheme. A center layer is composed of a β-APCN matrix containing a high drug loading and exhibiting high drug diffusivity and two outer layers which are also β-APCN-based, contain no-drug and are instead loaded with a diffusional barrier such as vitamin E, which considerably slows drug diffusion through these outer layers. Both modeling and experimental data demonstrates that the combined effect of non-uniform distribution of drug loading and diffusion constants within the three-layer systems of various embodiments of the present invention is capable of maintaining a low local drug concentration at the polymer-fluid interface, thus achieving zero-order kinetics.

### CROSS REFERENCE TO RELATED APPLICATIONS

This application claims the benefit of U.S. Provisional Patent Application Ser. No. 62/356,183 entitled “Zero-order Antibiotic Release from Multilayer Amphiphilic Conetwork Contact Lenses: Using On-Uniform Drug & Diffusivity Distributions for Constant-Rate Drug Delivery,” filed Jun. 29, 2016, and incorporated herein by reference in its entirety.

### FIELD OF THE INVENTION

One or more embodiments of the present invention relate to an apparatus and method for zero order drug delivery. In certain embodiments, the present invention relates to multilayer zero order drug delivery systems using the non-uniform drug and diffusivity distribution properties of bimodal amphiphilic co-network (β-APCN) matrices to generate constant-rate drug delivery.

### BACKGROUND OF THE INVENTION

Eye drops are almost universally used (˜90%) for the application of topical drugs to the eye. However, there are significant disadvantages to treating ocular conditions with eye drops. In the first place, the method is exceptionally inefficient, with only about 5% of the drug being absorbed while the rest enters the bloodstream through the conjunctival or the nasal path, causing considerable side-effects. The high drug waste necessitates a high drug concentration in the eye drops (up to ×600 times the therapeutic level), resulting in a sharp pulse of over-delivery followed by a long period of under delivery. In addition, eye drops must be administered multiple times per day, making patient compliance a significant issue. This is particularly critical for treating infections, especially in children and elderly population. Thus, a new approach that improves both compliance and bioavailability would be highly advantageous. Ophthalmic contact lenses have received a great deal of attention over the years, as they represent the best alternative to improve the delivery of topically applied drug solutions. The placement of the lenses on the cornea with limited mixing in the thin post lens tear film between the cornea and the lens, where drug molecules have a longer residence time compared to the case of topical application of eyedrops, has the potential of greatly increasing the bioavailability of the drug in the eye. Some estimations set the bioavailability from contact lenses to be as high as ˜50%, compared to 1-2% bioavailability from eyedrops.

Many of the current therapies for eye infections require a few days of drug delivery. In this way, a contact lens designed for such therapies should exhibit continuous release at the appropriate drug concentration for at least a few days. Unfortunately, it has been shown that commercial contact lenses release drugs for only a few hours. In these cases, a “burst” release is observed in which most of the drug is expelled in the first hour followed by several hours of low dose delivery. This type of release profile is characteristic of first order kinetic systems in which the release rate is diffusion controlled and concentration dependent (Fickian). As the drug concentration in the lens is at its highest at time zero, the rate of release is fastest and most of the drug is released in a burst.

Several approaches have been taken in order to increase the release time as well as to achieve zero-order (concentration-independent) release kinetics. One such approach involved a two-layer system in which an internal layer of PLGA was loaded with glaucoma drug during polymerization. The first lens was then “coated” by a second layer of pHEMA by submerging it in a monomer (HEMA) solution and using UV-light to polymerize the second layer around the first one. This system was able to provide glaucoma medication for over a month, but was unsuitable because of its poor oxygen permeability. A number of researchers have focused instead on developing biomimetic and ‘imprinted’ contact lenses, as well as nanoparticle loaded gels.

While these approaches were able to significantly increase the drug release duration from contact lenses, all the studies focused on hydrophilic hydrogel based contact lenses, which are not suitable for extended wear due to poor oxygen permeability. Current extended wear (highly oxygen permeable) contact lenses were made possible thanks to the independent discovery of Amphiphilic Polymer Conetworks in 1988 (known as Silicon-hydrogels in the contact lenses industry) by Kennedy in the US and Stadler in Germany. The conetworks incorporate co-continuous hydrophilic and hydrophobic phases, which allows for the incorporation of highly oxyphilic but otherwise hydrophobic moieties into a hydrogel (See FIG. 1).

Extensive work has also been done on therapeutic contact lenses using diffusion barriers, specifically vitamin E, to increase the drug release times for commercial silicon-hydrogel contact lenses. In-vitro and in-vivo experiments, supported by detailed modeling, have shown a great deal of success in increasing drug release times (up to a tenfold) from commercially available silicon-hydrogel lenses by the use of this technique.

The Higuchi equation has been used to model drug release for more than fifty years:

$$\frac{{M}_{t}}{{M}_{0}} = {k}^{′} {t}^{n} \qquad Eq . 1$$

where k′ is a rate constant characteristic of the system, Nₜ is the mass of drug released at time t, M₀ is the drug mass loaded in the system, t is time, and n an exponent characteristic of the mode of transport. For cases where n=0.5, the drug release follows the Fickian mechanism. For cases where n>0.5, anomalous (non-Fickian) diffusion is observed. The special case when n=1 gives rise to Case II transport mechanism. It is known that drug release from a system with a Case II type transport mechanism will be zero order. Even though the addition of vitamin E considerably reduces the effective diffusivity of the lenses, when approximating the release as square root kinetics using Eq. 1 above, the kinetics achieved with this technique were found to be Fickian (n=0.5), even for samples with very high vitamin E loading (˜60 wt %). The “burst” release is likely exacerbated via a non-uniform drug concentration distribution caused by convection with the water during the drying process (if the lenses are dried before release), leaving an uneven drug distribution across the lens, with higher concentrations at the surface.

Accordingly, what is needed in the art is a flexible β-APCN based drug delivery system that provides zero-order drug delivery kinetics, and have good oxygen permeability and translucency for therapeutic contact lens applications

### SUMMARY OF THE INVENTION

In one or more embodiments the present invention provides a novel approach to zero-order constant-rate drug delivery from therapeutic contact lenses and other systems using diffusion from amphiphilic conetworks. Quasi-Case II non-Fickian transport is achieved via non-uniform drug and diffusivity distributions within specially synthesized bimodal amphiphilic co-network ((3-APCN) matrices. In various embodiments, the system is based on a three-layer scheme. A center layer is composed of a β-APCN matrix containing a high drug loading and exhibiting high drug diffusivity and two outer layers which are also β-APCN-based, contain no-drug and are instead loaded with a diffusional barrier such as vitamin E that considerably slows drug diffusion through these outer layers. While single-layer neat-polymer and vitamin E loaded films displayed first order “burst” kinetics, both modeling and experimental data demonstrates that the combined effect of non-uniform distribution of drug loading and diffusion constants within the three-layer systems of various embodiments of the present invention is capable of maintaining a low local drug concentration at the polymer-fluid interface, thus achieving zero-order kinetics. As used herein, the terms “zero-order kinetics,” “zero-order release kinetics,” “zero-order drug delivery,” “zero-order constant-rate drug delivery,” or “zero-order release,” are used interchangeably to refer to drug release kinetics where the rate of drug release from the system does not depend upon the concentration of the drug loaded into the system. Drug release rates of topical antibiotics achieved by the various embodiments of the present invention are sufficient to provide constant-rate drug delivery at a therapeutic level with appropriate oxygen permeability for several days of extended wear.

In a first aspect, the present invention relates to a three layer bimodal amphiphilic co-network (β-APCN) based drug delivery device comprising: a middle β-APCN layer comprising a drug to be administered to a patient, the middle β-APCN layer having a first drug coefficient; a first and second outer β-APCN layer comprising a diffusional barrier material; each outer β-APCN layer having a first surface in contact with the middle β-APCN layer and a second surface in contact with a bodily fluid of the patient into whom the drug is to be delivered, wherein the first and second outer β-APCN layers have a second and third drug coefficient; wherein the first drug coefficient is larger than the second drug coefficient and the rate of drug release from the two outer β-APCN layers into the bodily fluid of the patient is substantially independent of the concentration of the drug in the middle β-APCN layer. In one or more embodiments, the middle β-APCN layer and the two outer β-APCN layers further comprise a co-network of poly(N,N-dimethylacrylamide) (PDMAAm) and polydimethylsiloxane (PDMS), crosslinked to form a β-APCN.

In one or more embodiments, the three layer β-APCN based drug delivery device of the present invention includes any one or more of the above referenced embodiments of the first aspect of the present invention wherein the drug to be administered to a patient is hydrophilic. In one or more embodiments, the three layer β-APCN based drug delivery device of the present invention includes any one or more of the above referenced embodiments of the first aspect of the present invention wherein the drug to be administered to a patient is selected from the group consisting of antibiotics, antimicrobials, antifungals, pain medications, steroids, and combinations thereof. In one or more embodiments, the three layer β-APCN based drug delivery device of the present invention includes any one or more of the above referenced embodiments of the first aspect of the present invention wherein the drug to be administered to a patient is selected from the group consisting of moxifloxacin hydrochloride, dexamethasone, levofloxacin, chlorhexidine, lidocaine, bupivacaine, tetracaine, cyclosporine A, timolol, dexamethasone 21-disodium phosphate, fluconazole, ofloxacin, and combinations thereof.

In one or more embodiments, the three layer β-APCN based drug delivery device of the present invention includes any one or more of the above referenced embodiments of the first aspect of the present invention wherein the ration of the first drug coefficient to the second and/or third drug coefficient is greater than 1:1, but not more than about 20:1. In one or more embodiments, the three layer β-APCN based drug delivery device of the present invention includes any one or more of the above referenced embodiments of the first aspect of the present invention wherein the diffusional barrier material is vitamin-E. In one or more embodiments, the three layer β-APCN based drug delivery device of the present invention includes any one or more of the above referenced embodiments of the first aspect of the present invention having a hydrophilic pore size of from about 30 nm to about 50 nm. In one or more embodiments, the three layer β-APCN based drug delivery device of the present invention includes any one or more of the above referenced embodiments of the first aspect of the present invention comprising a therapeutic contact lens.

In one or more embodiments, the three layer β-APCN based drug delivery device of the present invention includes any one or more of the above referenced embodiments of the first aspect of the present invention wherein the first outer β-APCN layer is substantially impermeable to the drug and need not have a second surface in contact with the bodily fluid of the patient; and substantially all of the drug is released through the second outer β-APCN layer. In one or more embodiments, the three layer β-APCN based drug delivery device of the present invention includes any one or more of the above referenced embodiments of the first aspect of the present invention comprising a wound dressing.

In a second aspect, the present invention relates to a method of making the three layer β-APCN based drug delivery device described above comprising: preparing the middle β-APCN layer and allowing it to dry; preparing a biocompatible solution comprising a drug to be delivered to a patient; loading the drug into the middle β-APCN layer by placing it into the biocompatible solution comprising a drug, whereby the drug is absorbed into the middle β-APCN layer; preparing a first outer β-APCN layer comprising a diffusional barrier material and a second outer β-APCN layer comprising a barrier material; the diffusional barrier material slowing the rate of diffusion of the drug through the first and second outer β-APCN layers; placing the middle β-APCN layer between the first and second outer β-APCN layers; and joining the first outer β-APCN layer, the middle β-APCN layer and the second outer β-APCN layer together to form the three layer β-APCN based drug delivery device of claim 1. In one or more of these embodiments, the middle β-APCN layer and the first and second outer β-APCN layers comprise a co-network of poly(N,N-dimethylacrylamide) (PDMAAm) and polydimethylsiloxane (PDMS), crosslinked to form a β-APCN.

In one or more embodiments, the method of making the three layer β-APCN based drug delivery device of the present invention includes any one or more of the above referenced embodiments of the second aspect of the present invention wherein the drug to be delivered to the patient is selected from the group consisting of antibiotics, antimicrobials, antifungals, pain medications, steroids, and combinations thereof. In one or more embodiments, the method of making the three layer β-APCN based drug delivery device of the present invention includes any one or more of the above referenced embodiments of the second aspect of the present invention wherein the drug to be delivered to the patient is selected from the group consisting of moxifloxacin hydrochloride, dexamethasone, levofloxacin, chlorhexidine, lidocaine, bupivacaine, tetracaine, cyclosporine A, timolol, dexamethasone 21-disodium phosphate, fluconazole, ofloxacin, and combinations thereof. In one or more embodiments, the method of making the three layer β-APCN based drug delivery device of the present invention includes any one or more of the above referenced embodiments of the second aspect of the present invention wherein the barrier materials comprises vitamin-E. In one or more embodiments, the method of making the three layer β-APCN based drug delivery device of the present invention includes any one or more of the above referenced embodiments of the second aspect of the present invention wherein the step of preparing the first and second outer β-APCN layers comprises adding the diffusional barrier material during formation of the β-APCN and before the final crosslinking of the polymer.

In a third aspect, the present invention is directed to a method of providing zero order drug release to a patient using the three layer β-APCN based drug delivery device of claim 1 comprising: preparing a three layer β-APCN based drug delivery device comprising: a middle β-APCN layer comprising a drug to be administered to a patient, the middle β-APCN layer having a first drug diffusion coefficient; a first and second outer β-APCN layer comprising a diffusion barrier material; each outer β-APCN layer having a first surface in contact with the middle β-APCN layer and a second surface, wherein the first and second outer β-APCN layers have a second and third drug coefficient; wherein the first drug coefficient is larger than the second and/or third drug diffusion coefficient; and placing the three layer β-APCN based drug delivery device into the bodily fluid of the patient into which the drug is to be delivered so that the second surfaces of the two outer β-APCN layers are in contact with the bodily fluid of the patient into which the drug is to be delivered, wherein the drug diffuses out of the second surfaces of the first and second outer β-APCN layers and into the bodily fluid of the patient at a rate that is substantially independent of the concentration of the drug loaded into the middle β-APCN layer. In one or more of these embodiments, the ratio of the first drug coefficient to the second and/or third drug coefficient is greater than 1:1 but not greater than about 20:1.

In one or more embodiments, the method of providing zero order drug release of the present invention includes any one or more of the above referenced embodiments of the third aspect of the present invention wherein the bodily fluid of the patient comprises, tears, blood, serum, interstitial fluid, spinal fluid, sweat, saliva or combinations thereof. In one or more embodiments, the method of providing zero order drug release of the present invention includes any one or more of the above referenced embodiments of the third aspect of the present invention wherein the three layer β-APCN based drug delivery device is a therapeutic contact lens. In one or more embodiments, the method of providing zero order drug release of the present invention includes any one or more of the above referenced embodiments of the third aspect of the present invention wherein the step of placing the three layer β-APCN based drug delivery device into the bodily fluid of the patient comprises placing the three layer β-APCN based drug delivery device between eyelid and cornea of the patient.

In one or more embodiments, the method of providing zero order drug release of the present invention includes any one or more of the above referenced embodiments of the third aspect of the present invention wherein the first outer β-APCN layer is substantially impermeable to the drug and need not have a second surface in contact with the bodily fluid of the patient; and substantially all of the drug is released through the second outer β-APCN layer. In one or more embodiments, the method of providing zero order drug release of the present invention includes any one or more of the above referenced embodiments of the third aspect of the present invention wherein the three layer β-APCN based drug delivery device is a wound dressing. In one or more embodiments, the method of providing zero order drug release of the present invention includes any one or more of the above referenced embodiments of the third aspect of the present invention wherein the step of placing the three layer β-APCN based drug delivery device into the bodily fluid of the patient comprises placing the three layer β-APCN based drug delivery device over a wound such that the second surface of the second outer β-APCN layer is in contact with the wound.

### BRIEF DESCRIPTION OF THE DRAWINGS

For a more complete understanding of the features and advantages of the present invention, reference is now made to the detailed description of the invention along with the accompanying figures in which:

FIG. 1 is a schematic illustration amphiphilic conetworks according to one or more embodiments of the present invention.

FIG. 2 is a graph showing concentration profiles for neat, vitamin E loaded (10%) and triple layer samples according to one or more embodiments of the present invention.

FIGS. 3A-B provide a one-dimensional diffusion model of a three-layer structure according to one or more embodiments of the present invention. FIG. 3A—is a schematic illustration of the proposed three-layer system showing two layers with small values of diffusion coefficient located on both sides of a middle layer that is loaded with drug and possess a higher diffusion coefficient. FIG. 3B—shows the governing equations, initial and boundary conditions for each layer.

FIGS. 4A-B is a schematic illustration of the effect of vitamin E loading in the β-APCNs microstructure, showing drug passageways without vitamin E loading (FIG. 4A) and with vitamin E loading (FIG. 4B).

FIGS. 5A-B are schematic diagrams showing a top view (FIG. 5A) and side view (FIG. 5B) of a wound dressing according to one or more embodiments of the present invention.

FIG. 6 is a graph showing the apparent oxygen permeability of β-APCNs according to one or more embodiments of the present invention at different crosslinker ratios.

FIG. 7 is a graph showing the light transmission of a representative β-APCN grade according to one or more embodiments of the present invention at wavelengths of from 200 nm to about 1100 nm.

FIG. 8 is a graph showing moxifloxacin hydrochloride release profiles for β-APCNs according to one or more embodiments of the present invention at different crosslinker ratios.

FIG. 9 is a graph showing moxifloxacin hydrochloride release profiles for β APCNs according to one or more embodiments of the present invention with different % of HMW-PDMS.

FIG. 10 is a graph showing moxifloxacin hydrochloride release profiles for β-APCNs according to one or more embodiments of the present invention with different vitamin E loadings.

FIG. 11 is a graph showing concentration profiles for moxifloxacin hydrochloride release from for β-APCNs according to one or more embodiments of the present invention with different vitamin E loadings.

FIG. 12A-D are model release results for a three-layer drug release system according to one or more embodiments of the present invention showing concentration profiles as a function of dimensionless time and thickness for different D₁/D₂ ratios of 1 (FIG. 12A), 10 (FIG. 12B), 100 (FIG. 12C), and 1000 (FIG. 12D).

FIG. 13 is a graph of model release results for a three-layer drug release system according to one or more embodiments of the present invention showing concentration at the boundary as a function of dimensionless time for different D₁/D₂ ratios.

FIG. 14 is a graph of model release results for a three-layer drug release system according to one or more embodiments of the present invention showing concentration at the boundary as a function of dimensionless time for different K values.

FIG. 15 is a graph showing moxifloxacin hydrochloride release profiles for triple layer samples according to one or more embodiments of the present invention.

FIG. 16 is a graph showing % drugs release by triple layer samples according to one or more embodiments of the present invention as a function of the square root of time. The lines in the figure are the best fit straight line to short-time release data.

FIG. 17 is a schematic representation of the non-uniform drug concentration distribution within the samples and the difference in diffusion constants between the middle and outer layers of three-layer drug delivery systems according to one or more embodiments of the present invention.

FIG. 18 is a chart showing gel permeation chromatography (GPC) traces for a) V-PDMS-MA, b) [PDMAAm(PDMS)]-g-PDMS-V-0, c) [PDMAAm(PDMS)]-g-PDMS-V-1, d) [PDMAAm(PDMS)]-g-PDMS-V -2, e) [PDMAAm(PDMS)]-g-PDMS-V-5 for comparison.

### DETAILED DESCRIPTION OF THE ILLUSTRATIVE EMBODIMENTS

In one or more embodiments, the present invention provides a zero-order constant-rate drug delivery system using specially synthesized bimodal amphiphilic conetwork (β-APCN) matrices to provide Quasi-Case II non-Fickian transport via non-uniform drug and diffusivity distributions within the specially synthesized bimodal amphiphilic conetwork (β-APCN) matrices. In various embodiments, the system is based on a three-layer scheme having a center layer is composed of a β-APCN matrix that contains a high drug loading and exhibits high drug diffusivity and two outer layers which are also β-APCN-based, but contain no-drug and are instead loaded with a diffusional barrier, such as vitamin E, which considerably slows drug diffusion. While single-layer neat-polymer β-APCN and vitamin E loaded β-APCN films displayed first order “burst” kinetics, both modeling and experimental data demonstrate that the combined effect of non-uniform distribution of drug loading and diffusion constants within the three-layer system is capable of providing and maintaining a low local drug concentration at the polymer-fluid interface, thus achieving zero-order kinetics.

In one or more embodiments of the drug delivery system of the present invention, non-Fickian kinetics is achieved via a non-uniform drug concentration distribution within the system and a significant difference in diffusion coefficients between the middle and outer layers. Drug release rates of topical antibiotics achieved by the drug delivery system of the present invention are sufficient to provide constant-rate drug delivery at a therapeutic level. For contact lens embodiments, these systems were also shown to be translucent for visible wavelengths of light and to provide appropriate oxygen permeability for several days of extended wear.

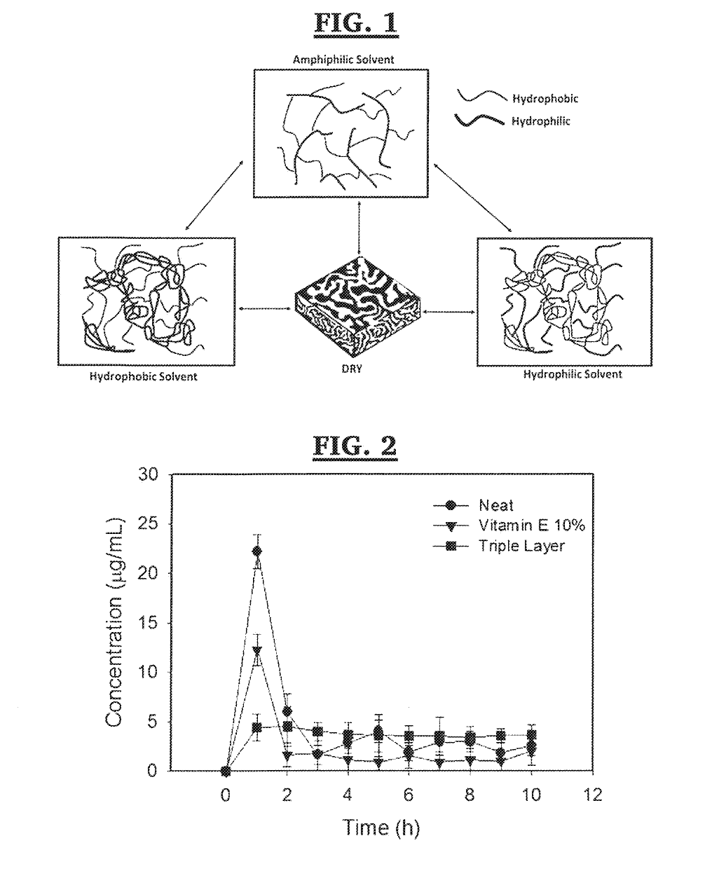

As will be appreciated by those of skill in the art, an amphiphilic co-network (APCN) is a polymer network that includes a hydrophobic constituent and a hydrophilic constituent that are interconnected to create a co-continuous morphology of hydrophobic phases and a hydrophilic phases. This, in turn, allows amphiphilic co-networks to have both hydrophobic pores/channels and hydrophilic pores/channels, permitting the amphiphilic co-network to bipercoluate. (See FIG. 1). As used herein, the term bipercoluate refers to the ability of a material to allow solvents of different polarities, such as water and a hydrocarbon, to permeate separately from edge to edge of the entire amphiphilic co-network. (See, e.g., FIGS. 1, 2). Put another way, these amphiphilic co-networks may be a hydrogel that swells in both hydrophilic solvents like water and hydrophobic solvents like hydrocarbons.

As used herein, the term bimodal amphiphilic co-network (β-APCN) refers to an amphiphilic co-network having a mixture of high and low molecular weight chains. The bimodal nature of these co-networks both greatly enhances their mechanical properties and provides an additional means of controlling their functional properties. As should be apparent and will be discussed in greater detail below, the presence of both high and low molecular weight chains in either or both constituents will tend to create pores/channels in and/or through that constituent of the β-APCN when it is swollen. For example, when a β-APCN is submerged in water, the hydrophilic phase swells, creating water-swollen-channels for diffusion of hydrophilic molecules (the opposite occurs in a non-water environment). The diameter of such channels may be controlled by controlling the relative ratio of hydrophilic/hydrophobic chains and the mesh size. The mesh size may, in some embodiments, be tuned by controlling the ratio of high to low molecular weight chains and/or the crosslinker ratio. Bimodal Amphiphilic co-networks (β-APCN) provide a unique route to integrate contrasting attributes of otherwise immiscible components within a single material. This characteristic allows them to exhibit unique properties and makes them exceptional materials for therapeutic contact lenses and wound dressings, among other things.

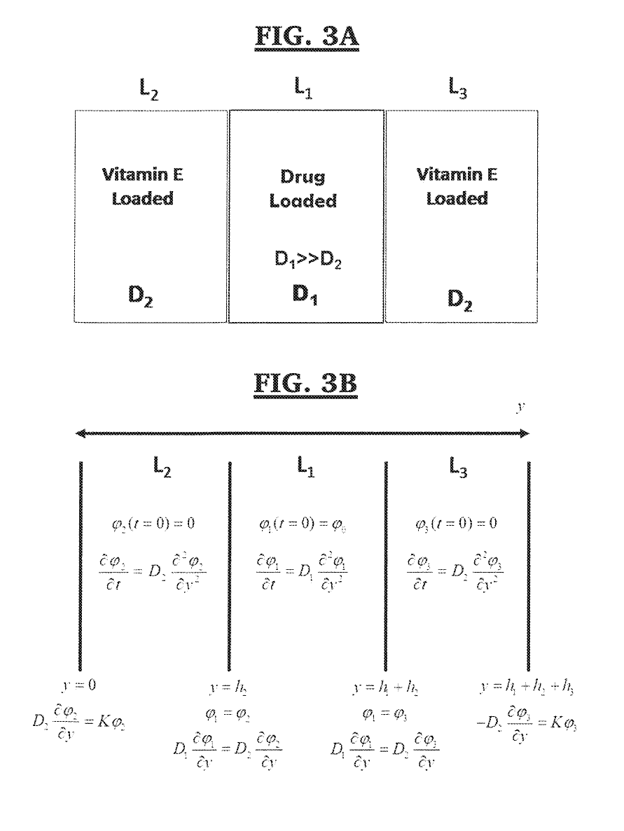

As set forth above and shown in FIG. 3, in various embodiments, the drug delivery device of the present invention comprises three layers—a β-APCN middle or inner layer (L2) having relatively high diffusivity and containing the drug to be delivered to the patient, and two β-APCN outer layers (L1, L3) that include one or more diffusion barriers that slow diffusion of the drug as it passes from the inner layer (L2), through the outer layers (L1 and L3) to the bodily fluid of the patient into which the drug is to be delivered. The bodily fluid of the patient into which the drug is to be delivered is not particularly limited and may be any fluid in the patient's body including, but not limited to, tears, blood, serum, interstitial fluid, spinal fluid, sweat, saliva or combinations thereof. Once the drug reaches the polymer-fluid interface, is quickly removed (absorbed by the patient) under sink conditions. In this manner, the concentration at the polymer-fluid layer interface is low when the diffusion begins, thus preventing a “burst” release.

The rate at which the drug diffuses through a material may be described in terms of its diffusion coefficient (D). The diffusion coefficient (D) may be calculated from the following equation:

$$\frac{\partial C}{\partial t} = D \frac{{\partial }^{2} c}{\partial {y}^{2}} \qquad ( Eq . 2 )$$

where C is the drug concentration in the β-APCN, D is the effective diffusivity (diffusion coefficient) of the material (i.e. the β-APCN layer being evaluated) and y and t denote the transverse coordinate and time, respectively. Eq. 2 makes the reasonable assumptions that the surrounding fluid volume is much larger than β-APCN volume, the drug is uniformly distributed in the β-APCN layer and the solubility of the drug is very high in the surrounding media and is applicable for any Fickian diffusion system of these conditions. When setting the proper boundary conditions under perfect sink, and solving Eq. 2, it is possible to obtain a time function for both the concentration profile in the β-APCN layers and the concentration in the release media. (See e.g., FIG. 3B) The concentration in the release media is found to be a linear function of the initial concentration and a square root function of time and the diffusion coefficient, C(D^{0.5},t^{0.5}, Cᵢ). The result is similar to the Fickian case of the Higuchi equation (Eq. 1)

In the various embodiments of the present invention, the diffusion coefficient of the inner layer (L2) (referred to herein as D1) is significantly larger than the diffusion coefficient of the outer layers (L2) (referred to herein as D1). See FIG. 3A. In one or more embodiments, the ratio of Dl to D2 is from about 1.1:1 to about 1200:1. In some embodiments, the ratio of D1 to D2 is 4:1 or greater, in other embodiments, 10:1 or greater, in other embodiments, 50:1 or greater, in other embodiments, 100:1 or greater, in other embodiments, 500:1 or greater, in other embodiments, 700:1 or greater and, in other embodiments, 1000:1 or greater. The ration may, in some other embodiments, in excess of 1200:1. In some embodiments, the ratio of D1 to D2 is 1100:1 or less, in other embodiments, 1000:1 or less, in other embodiments, 800:1 or less, in other embodiments, 600:1 or less, in other embodiments, 500:1 or less, in other embodiments, 400:1 or less, in other embodiments, 300:1 or less and , in other embodiments, 200:1 or less. In some embodiments, the ratio of Dl to D2 is about 4.5:1.

There are a variety of factors that may affect diffusion of a drug through the various β-APCN layers of the drug delivery device of the present invention. These factors include, but are not limited to: the specific type of β-APCN; the β-APCN concentration in the swelled matrix; the ratio of hydrophilic to hydrophobic constituents in the β-APCN; the ratio of low molecular weight to high molecular weight chains forming the β-APCN; the degree to which the β-APCN is crosslinked; the pore size of the β-APCN; the type of and amount of barrier material, if any; and the length of the drug diffusion path.

Any suitable β-APCN, including commercially available silicon hydrogels (SiH), may be used to practice one or more embodiments of the present invention provide that they exhibit sufficient oxygen permeability (necessary for extended wear) and can loaded with both hydrophilic and hydrophobic drugs, and a diffusion barrier material such as vitamin E. Traditional hydrogel materials that do not contain siloxanes (pHEMA, PEG, etc.) are not suitable for extended delivery applications due to their low oxygen permeability.

Suitable β-APCNs may include those disclosed in J. Kim, C.C. Peng, A. Chauhan, “Extended release of dexamethasone from silicone-hydrogel contact lenses containing vitamin E,” J. Control. Release, 148 (2010) 110-116 (doi:10.1016/j.jconre1.2010.07.119); P. Paradiso, A. P. Serro, B. Saramago, R. Colaco, A. Chauhan, “Controlled Release of Antibiotics From Vitamin E-Loaded Silicone-Hydrogel Contact Lenses,” J. Pharm. Sci., 3549 (2016) 1-9 (doi:10.1016/50022-3549(15)00193-8); C.C. Peng, M. T. Burke, A. Chauhan, “Transport of topical anesthetics in vitamin e loaded silicone hydrogel contact lenses,” Langmuir, 28 (2012) 1478-1487, (doi:10.1021/1a203606z); C. C. Peng, A. Chauhan, “Extended cyclosporine delivery by silicone-hydrogel contact lenses,” J. Control. Release, 154 (2011) 267-274. (doi:10.1016/j.jconre1.2011.06.028); C. C. Peng, J. Kim, A. Chauhan, “Extended delivery of hydrophilic drugs from silicone-hydrogel contact lenses containing Vitamin E diffusion barriers,” Biomaterials, 31 (2010) 4032-4047 (doi:10.1016/j.biomaterials.2010.01.113), the disclosures of which are incorporated herein by reference in their entirety. Suitable β-APCNs may include, without limitation, bimodal co-networks of poly(N,N-dimethylacrylamide) (PDMAAm) and polydimethylsiloxane (PDMS), crosslinked to form a β-APCN. In one or more embodiments of the present invention, the β-APCN of the present invention may be any of the crosslinked bimodal graft APCNs described in G. Guzman, T. Nugay, l. Nugay, N. Nugay, J. Kennedy, M. Cakmak, Macromolecules 2015, 48, 6251 and/or International Published Patent Application No. WO 2014/197699, the disclosures of which are incorporated herein by reference in their entirety.

In various embodiments, the β-APCN used for the drug delivery device of the present invention may be formed by crosslinking a molecularly-bimodal crosslinkable amphiphilic graft, which includes a hydrophobic constituent and a hydrophilic constituent. In these embodiments, the hydrophilic constituent forms a backbone carrying hydrophobic branches. Each branch may include a crosslinkable end group. The molecularly-bimodal crosslinkable amphiphilic graft may include first set of hydrophobic branches and a second set of hydrophobic branches, were the second set of hydrophobic branches has a substantially longer chain length. In one or more embodiments, the molecularly-bimodal crosslinkable amphiphilic graft may be soluble (e.g. in THF). The molecularly-bimodal crosslinkable amphiphilic graft may then be crosslinked to from a β-APCN.

In some of these embodiments, these molecularly-bimodal crosslinkable amphiphilic grafts may be prepared by polymerizing a dihydrocarbylacrylamide monomer in the presence of a first asymmetric-telechelic polydihydrocarbylsiloxane monomer mixture and a second asymmetric-telechelic polydihydrocarbylsiloxane monomer mixture. As used herein, the term “asymmetric-telechelic polydihydrocarbylsiloxane monomer mixture” refers to a mixture of polydihydrocarbylsiloxane monomers that include two different terminal functional groups that allow for further reaction or polymerization. In one or more of these embodiments, the asymmetric-telechelic polydihydrocarbylsiloxane monomer mixture may include a polydihydrocarbylsiloxane monomer (PDHS), with two first terminal functional groups (A) in a telechelic monomer (A-PDHS-A), a polydihydrocarbylsiloxane monomer, with two second terminal functional groups (B) in a telechelic monomer (B-PDHS-B), and a polydihydrocarbylsiloxane monomer, with first terminal functional groups and a second terminal functional groups in a di-end-functional monomer (A-PDHS-B).

In one or more of these embodiments, the molar mass ratio between average molar mass of the monomers in the first asymmetric-telechelic monomer polydihydrocarbylsiloxane mixture and average molar mass of the monomers in the second asymmetric-telechelic monomer polydihydrocarbylsiloxane mixture is between about 1:2 and about 1:20, in other embodiments between about 1:8 and about 1:15, in other embodiments between about 1:4 and about 1:10, and in other embodiments between about 1:5 and about 1:8. In one or more embodiments, the second asymmetric-telechelic polydihydrocarbylsiloxane monomer mixture is 0.1% to 10%, in other embodiments 0.5% to 7%, and in other embodiments 1% to 5% of the total asymmetric-telechelic polydihydrocarbylsiloxane monomer. The total polydihydrocarbylsiloxane monomer is the sum of all of the polydihydrocarbylsiloxane monomer mixtures.

In some of these embodiments, both the asymmetric-telechelic polydihydrocarbylsiloxane monomer mixtures include:

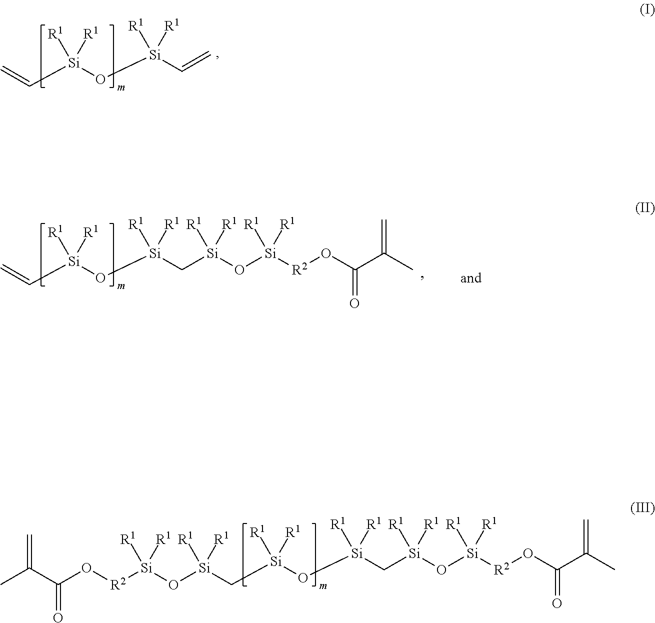

where each R¹ is individually a monovalent organic group, each R² is individually a divalent organic group. For the first asymmetric-telechelic monomer polydihydrocarbylsiloxane mixture, each m group is individually an integer from about 100 to about 500, in other embodiments from about 180 to about 350, in other embodiments from about 190 to about 320, in other embodiments from about 195 to about 315. For the second asymmetric-telechelic monomer polydihydrocarbylsiloxane mixture, each m group is an integer from about 1000 to about 2000, in other embodiments from about 1050 to about 1950, in other embodiments from about 1100 to about 1900, in other embodiments from about 1150 to about 1850.

The first asymmetric-telechelic polydihydrocarbylsiloxane monomer mixture and the second asymmetric-telechelic polydihydrocarbylsiloxane monomer mixture may be prepared together or separately. In some of these embodiments, the asymmetric-telechelic polydihydrocarbylsiloxane monomer mixture is prepared by reacting a vinyl telechelic polydihydrocarbylsiloxane with a disiloxane acrylate. A telechelic polydihydrocarbylsiloxane to disiloxane acrylate molar ratio of less than 1:2 is used to produce an asymmetric-telechelic polydihydrocarbylsiloxane monomer mixture that includes asymmetric-telechelic polydihydrocarbylsiloxane monomer vinyl and acrylate end groups. In one or more embodiments, the telechelic polydihydrocarbylsiloxane to disiloxane acrylate molar ratio less than 1:2, in other embodiments less than 1:1.5, in other embodiments less than 1:1. In one or more embodiments, where the first asymmetric-telechelic polydihydrocarbylsiloxane monomer mixture and the second asymmetric-telechelic polydihydrocarbylsiloxane monomer mixture are prepared together, a first vinyl telechelic polydihydrocarbylsiloxanes and a second vinyl telechelic polydihydrocarbylsiloxane with a longer chain length are reacted with a disiloxane acrylate in the same reaction mixture.

The reaction between a telechelic polydihydrocarbylsiloxane and the disiloxane acrylate is a hydrosylation reaction. In one or more embodiments a platinum catalyst may be used to facilitate the hydrosylation telechelic polydihydrocarbylsiloxane and the disiloxane acrylate. Suitable platinum catalysts include Karstedt's catalysts.

The first asymmetric-telechelic polydihydrocarbylsiloxane monomer mixture and the second asymmetric-telechelic polydihydrocarbylsiloxane monomer mixture may be combined with a dihydrocarbylacrylamide monomer. The dihydrocarbylacrylamide monomer may polymerized to prepare the molecularly-bimodal crosslinkable amphiphilic graft. The polymerization may take place under free radical conditions. In one or more embodiments the dihydrocarbylacrylamide monomer may be between about 40 wt % and about 70 wt %, in other embodiments about 45 wt % and about 65 wt %, and in other embodiments about 50 wt % and about 60 wt % of the total weight of the dihydrocarbylacrylamide monomer, first asymmetric-telechelic polydihydrocarbylsiloxane monomer mixture, and second asymmetric-telechelic polydihydrocarbylsiloxane monomer mixture.

In one or more embodiments, the dihydrocarbylacrylamide monomer may be defined by the formula

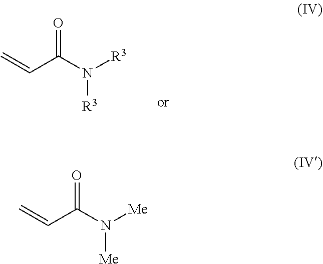

where each R³ is individually a monovalent organic group.

In these embodiments, the molecularly-bimodal crosslinkable amphiphilic graft is crosslinked with a siloxane compound that includes at least two Si—H bonds via a hydrosylation reaction. In one or more of these embodiments a platinum catalyst, such as Karstedt's catalysts, may be used to facilitate the hydrosylation of the telechelic polydihydrocarbylsiloxane and the disiloxane acrylate. In these embodiments, the amount of siloxane compound that includes at least two Si—H bonds may be characterized in reference to the amount of vinyl end groups present in the molecularly-bimodal crosslinkable amphiphilic graft. In one or more embodiments, the vinyl group to Si—H bond ratio is about 1:1 to about 1:30, in other embodiments about 1:3 to about 1:25, and in other embodiments about 1:5 to about 1:10.

In one or more of these embodiments, the amount of siloxane compound that includes at least two Si—H bonds may be characterized by the percent weight of the siloxane compound that includes at least two Si—H bonds out of the total of the molecularly-bimodal crosslinkable amphiphilic graft and the siloxane compound that includes at least two Si—H bonds. In one or more embodiments, the percent weight of the siloxane compound that includes at least two Si—H bonds is from about 1% to about 30%, in other embodiments from about 3 to about 25%, and in other embodiments about 5% to about 10% of the total of the molecularly-bimodal crosslinkable amphiphilic graft and the siloxane compound that includes at least two Si—H bonds. Suitable siloxane compounds that includes at least two Si—H bonds for crosslinking the crosslinkable amphiphilic graft may be found in U.S. Pat. Nos. 8,247,515 and 8,067,521, both of which are incorporated by reference. In one or more embodiments, the siloxane compound that includes at least two Si—H bonds may be defined by the formula

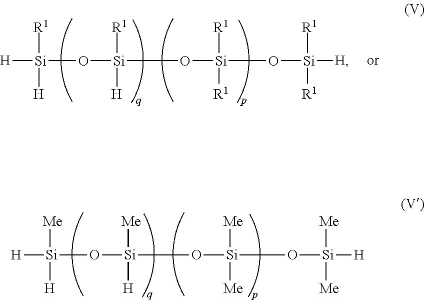

where each R¹ is individually a monovalent organic group, and p and q are each an integer from about 1 to about 2000. In one or more embodiments, p is an integer from 1 to 1000, in other embodiments, from 1 to 500, in other embodiments, from 3 to 100, in other embodiments, 5 to 50, and in other embodiments, 10 to 30. In one or more embodiments, q is an integer from 1 to 1000, in other embodiments, from 1 to 500, in other embodiments, from 3 to 100, in other embodiments, 5 to 50, and in other embodiments, 10 to 30.

Unless otherwise indicated, number average molecular weights referenced herein, may be determined using any appropriate method known in the art, including without limitation, size exclusion chromatography (SEC), mass spectroscopy or other known measuring technique. Likewise, unless otherwise indicated, any weight average molecular weights referenced herein, may be determined using any appropriate method known in the art, including without limitation, size exclusion chromatography (SEC), mass spectroscopy, or other known measuring technique. In one or more embodiment, the number average molecular weight of PDMS for β-APCN used to form the drug delivery device of the present invention is 10 kDa or more, in other embodiments, 20 or more, in other embodiments, 30 kDa or more, in other embodiments, 40 kDa or more, in other embodiments, 50 kDa or more, in other embodiments, 60 kDa or more, and in other embodiments, 70 kDa or more. In one or more embodiment, the number average molecular weight of PDMS for the β-APCN used to form the drug delivery device of the present invention is 150 kDa or less, in other embodiments, 120 kDa or less, in other embodiments, 90 kDa or less, in other embodiments, 80 kDa or less, in other embodiments, 70 kDa or less, in other embodiments, 60 kDa or less, in other embodiments, 50 kDa or less, and in other embodiments, 40 kDa or less.

As will be apparent to those of skill in the art, the relative concentrations of polymer and water in the swelled β-APCN matrix will affect the diffusion properties of the β-APCN layers of the present invention. All other things being equal, the higher the concentration of polymer in the β-APCN matrix forming the β-APCN layers of the present invention, the less room there is for the formation of pores and channels for diffusion. Conversely, the higher the water content in the swelled β-APCN matrix the more room there is for the formation of pores and channels for diffusion. The water content in the swelled material β-APCN matrix will depend on the relative ratio between hydrophilic and hydrophobic phases and on the mesh size (as controlled by the crosslinker ratio and the ratio of low to high molecular weight polymer chains).

It should also be appreciated that the relative weight percent of β-APCN in the swelled β-APCN co-network will depend both on the composition of the β-APCN and the density of the liquid material used to swell the β-APCN matrix. In one or more embodiments, the β-APCNs used for the invention may have a water content of from about 40 weight percent to about 70 weight percent, but it should be appreciated that lower or higher water contents are possible by changing the overall ratio of hydrophilic to hydrophobic chains in the β-APCN used. In one or more embodiments, the β-APCNs used for the invention may have a water content of about 43 weight percent, or more, in other embodiments, 45 weight percent or more, in other embodiments, 47 weight percent or more, in other embodiments, 50 weight percent or more, in other embodiments, 55 weight percent or more, and in other embodiments, 60 weight percent or more. In one or more embodiments, the β-APCNs used for the invention may have a water content of about 73 weight percent or less, in other embodiments, 68 weight percent or less, in other embodiments, 65 weight percent or less, in other embodiments, 60 weight percent or less, in other embodiments, 55 weight percent or less, in other embodiments, 50 weight percent or less. It should also be noted, however, that particularly for contact lens based embodiments, a very high water content would significantly reduce oxygen permeability and a very low content would compromise ion permeability and comfort.

As set forth above, the ratio of hydrophilic constituents to hydrophobic constituents in the β-APCN may affect the diffusions properties of the β-APCN layers of the present invention. For example, if the drug to be distributed is hydrophilic, a higher ratio of hydrophilic constituents to hydrophobic constituents will provide more pores and channels for diffusion of the hydrophilic drug and a low ratio of hydrophilic constituents to hydrophobic constituents will provide fewer. Conversely, if the drug to be distributed is hydrophobic, a higher ratio of hydrophobic constituents to hydrophilic constituents will provide more pores and channels for diffusion of the hydrophobic drug and a low ratio of hydrophobic constituents to hydrophilic constituents will provide fewer.

In one or more embodiments, the ratio of hydrophilic constituents to hydrophobic constituents in the β-APCN layers of the present invention may be from about 1:4 to about 4:1. In some embodiments, the ratio of hydrophilic constituents to hydrophobic constituents in the β-APCN layers of the present invention may be 1:3.5 or more, in other embodiments, 1:3 or more, in other embodiments, 1:2.5 or more, in other embodiments, 1:2 or more, in other embodiments, 1:1.5 or more, in other embodiments, 1:1.25 or more and, in other embodiments, 1:1 or more. In some embodiments, the ratio of hydrophilic constituents to hydrophobic constituents in the β-APCN layers of the present invention may be 3.5:1 or less, in other embodiments, 3:1 or less, in other embodiments, 2.5:1 or less, in other embodiments, 2:1 or less, in other embodiments, 1.5:1 or less, in other embodiments, 1.25:1 or less and, in other embodiments, 1:1 or less.

As set forth above, the hydrophilic and/or hydrophobic constituents of the β-APCNs used to form the three layer drug delivery device of the present invention may be formed from a mixture of high and low molecular weight polymer chains. As will be appreciated by those of skill in the art, the relative amount of high and low molecular weight polymer chains in the hydrophilic and/or hydrophobic constituents of the β-APCNs will affect the diffusion properties of the β-APCN layers of the present invention. In one or more embodiments, mole percentage of the longer high molecular weight polymer chains in the β-APCN used in the present invention is from about 1% to about 8%. Conversely, in one or more embodiments, mole percentage of the shorter low molecular weight polymer chains in the β-APCN used in the present invention is from about 92% to about 99%.

While high molecular weight and low molecular weight are relative terms, it is the relative difference in chain length between the longer high molecular weight polymer chains and the shorter low molecular weight polymer chains, rather than the specific value of each, that affects mesh size and with it, the diffusion properties of the β-APCN layers of the present invention. In some embodiments, the ratio of the number average molecular weight of the high molecular weight polymer chains to number average molecular weight of the low molecular weight polymer chains may be 2:1 or greater, in other embodiments, 4:1 or greater, in other embodiments, 6:1 or greater, in other embodiments, 8:1 or greater, in other embodiments, 10:1 or greater, in other embodiments, 12:1 or greater, and in other embodiments, 14:1 or greater. In some embodiments, the ratio of the number average molecular weight of the high molecular weight polymer chains to the number average molecular weight of the low molecular weight polymer chains may be 20:1 or less, in other embodiments, 19:1 or less, in other embodiments, 17:1 or less, in other embodiments, 16:1 or less, in other embodiments, 15:1 or less, in other embodiments, 13:1 or less, and in other embodiments, 11:1 or less. It is expected that the ratio of the number average molecular weight of the high molecular weight polymer chains to the number average molecular weight of the low molecular weight polymer chains will be from about 4:1 to about 10:1.

In some embodiments, the low molecular weight polymer chains used to form the β-APCN layers of the present invention may be a PDMS polymer mixture having a number average molecular weight of from about 6 kDa to about 15 kDa, in other embodiments, from about 10 kDa to about 15 kDa in other embodiments, from about 6 kDa to about 10 kDa, and in other embodiments, from about 8 kDa to about 12 kDa. In some embodiments, the high molecular weight polymer chains used to form the β-APCN layers of the present invention may be a PDMS polymer mixture having a number average molecular weight of from about 60 kDa to about 100 kDa, in other embodiments, from about 75 kDa to about 100 kDa, in other embodiments, from about 60 kDa to about 85 kDa, and in other embodiments, from about 70 kDa to about 90 kDa.

It should also be appreciated that for the β-APCN to be formed, all constituents (hydrophilic and hydrophobic) must be part of a single crosslinked network. In some of these embodiments, the network formation is carried in two steps: first is the synthesis of an amphiphilic graft polymer in which PDMS chains are linked to PDMAAm chains; and second, those amphiphilic grafted PDMS chains are crosslink with themselves using a PDMS-based crosslinker to form a single crosslinked network.

As set forth above, the degree to which the β-APCNs are crosslinked will also affect the diffusions properties of the β-APCN layers of the drug delivery device of the present invention. In some of these co-networks, substantially all of the hydrophobic constituents and hydrophilic constituents are crosslinked. It has also been found that crosslinks in the hydrophobic phase contract the network and that an increased crosslinker ratio increases drug release time for hydrophilic drugs. As set forth in more detail below, samples with a higher crosslinker ratio presented considerably slower release kinetics. The same effect is observed in samples with a higher percentage of high molecular weight (long chain) monomer, albeit less pronounced. Further, the molecular weight of the short monomer chains may be below the entanglement molecular weight of the polymer, while that of the long monomer chains is considerably above this value. In this manner, the number of entanglements considerably increases by increasing the content of long polymer chains in the network. It has been found that some fraction of these entanglements present in the bulk polymer before cross-linking become permanently trapped during network formation and act as additional cross-links, further constraining the network and increasing drug release times.

In one or more embodiments, the β-APCN of the drug delivery device of the present invention may have a molar ratio of chain ends for bonding (e.g. allyl chain ends) to crosslinker (e.g. hydrosilixane) (referred to herein as the “crosslinker ratio”) of from about 1:5 to about 1:25, in other embodiments, about 1:5 to about 1:10, in other embodiments, about 1:5 to about 1:25, in other embodiments, about 1:10 to about 1:25.

Further, as set forth above, the size of the pores or channels of the β-APCN through layers which the drug is to pass also affects the diffusions properties of the β-APCN layers of the drug delivery device of the present invention. As should be apparent, the pores or channels of the β-APCN must to large enough to permit the drug chosen to be loading into and then diffuse out of the swollen, β-APCN matrix. In the swollen state, a hydrophilic drug permeates only through hydrophilic channels formed by water-swollen hydrophilic domains. The dimensions of these channels are controlled by the molecular weight between crosslinks (M) of the hydrophilic moiety and by morphological thermodynamic/kinetic constrains on the network. The calculation of M_{c} is specific for a given network topology.

One of ordinary skill in the art will be able to determine the molecular weight between crosslinks (M_{c}) for a particular β-APCN without undue experimentation. In one or more embodiments, M_{c} for a β-APCN may be calculated by (i) determining the number average molecular weight and the number of polymer chains in the β-APCN; (ii) determining the number of crosslinking or other side chains per mole of polymer chains; and (iii) dividing number average molecular weight of the polymer chains by 1 plus the number of crosslinking or other side chains per polymer chain to arrive at the molecular weight between crosslinks (M) for the β-APCN. In one or more embodiments, M_{c} may be calculated as described in Example 2, below. This calculation is based on calculated M_{n PDMAAm} in case of 100% initiator efficiency (AIBN) and some experimental data (M_{n PDMS}, WPDMS and W_{PDMAAm}), which has been found to be the best method for calculating approximate value of M_{c}.

For the bimodal co-networks of crosslinked poly(N,N-dimethylacrylamide) (PDMAAm) and polydimethylsiloxane (PDMS) described above and in the Experimental section to follow, M_{c} was calculated by:

$${M}_{c} = \frac{( {M}_{n , PDMAAm} \cdot {M}_{n , MA \text{-} PDMS \text{-} V} \cdot {W}_{PDMAAm} )}{\begin{matrix}( {W}_{MA \text{-} PDMS \text{-} V} \cdot {M}_{n , PDMAAm} ) + \\ ( {M}_{n , MA \text{-} PDMS \text{-} V} \cdot {W}_{PDMAAm} )\end{matrix}} \qquad ( Eq . 3 )$$

where, W_{PDMAAm} and W_{MA-PDMS-V} are the weights of PDMAAm and acrylate end functionalized PDMS, respectively, M_{n,PDMAAm} is the number average molecular weight of PDMAAm determined by GPC, whereas M_{n,MA-PDMS-V} is that of acrylate end functionalized PDMS. In some other embodiments, the determination Mₙ of PDMAAm may be performed by homo-polymerization DMAAm under the same experimental conditions of the graft copolymerization and then GPC characterization. In the M_{c} calculation of Mₙ PDMAAm, GPC can be used instead of above mentioned calculated Mₙ PDMAAm.

In one or more embodiments, molecular weight between crosslinks (M) of the hydrophilic moiety may be from 3,000 g/mol to 30,000 g/mol. In some embodiments, the molecular weight between crosslinks (M) of the hydrophilic moiety may be 5,000 g/mol or more, in other embodiments, 7,500 g/mol or more, in other embodiments, 10,000 g/mol or more, in other embodiments, 15,000 g/mol or more and, in other embodiments, 20,000 g/mol or more. In some embodiments, the molecular weight between crosslinks (Mₑ) of the hydrophilic moiety may be 27,000 g/mol or less, in other embodiments, 25,000 g/mol or less, in other embodiments, 22,500 g/mol or less, in other embodiments, 20,000 g/mol or less, in other embodiments, 17,500 g/mol or less, and in other embodiments, 15,000 g/mol or less.

The size of hydrophilic pores and/or channels used for diffusion of hydrophilic drugs through the β-APCN used for the β-APCN based drug delivery device of the present invention is not particularly limited and can, in theory, have any hydrodynamic radius, provided that the hydrodynamic radius is large enough to permit passage of the drug to be delivered through the β-APCN. In various embodiments, the β-APCN used for the β-APCN based drug delivery device of the present invention will have hydrophilic pores and/or channels suitable for diffusion of a hydrophilic drug with a hydrodynamic radius of from about 0.001 nm to about 100nm. In some embodiments, the hydrodynamic radius may be 3 nm or more, in other embodiments, l0 nm or more, in other embodiments, 20 nm or more, in other embodiments, 30 nm or more, in other embodiments, 40 nm or more and, in other embodiments, 50 nm or more. In some embodiments, the hydrodynamic radius may be 90 nm or less, in other embodiments, 80 nm or less, in other embodiments, 70 nm or less, in other embodiments, 60 nm or less, in other embodiments, 50 nm or less, and in other embodiments, 40 nm or less. In various embodiments, the β-APCN used for the β-APCN based drug delivery device of the present invention will have hydrophilic pores and/or channels suitable for diffusion of a hydrophilic drug with a hydrodynamic radius of from about 30 nm to about 50 nm.

In various embodiments, the β-APCN used for the β-APCN based drug delivery device of the present invention will have hydrophobic pores and/or channels suitable for diffusion of a hydrophobic drug that have a hydrodynamic radius of from about 0.001 nm to about 100 nm. In some embodiments, the radius may be 5 or more, in other embodiments, 10 nm or more, in other embodiments, 20 nm or more, in other embodiments, 30 nm or more and, in other embodiments, 40 nm or more. In some embodiments, the radius may be 80 nm or less, in other embodiments, 60 nm or less, in other embodiments, 50 nm or less, in other embodiments, 40 nm or less, in other embodiments, 30 nm or less, and in other embodiments, 20 or less.

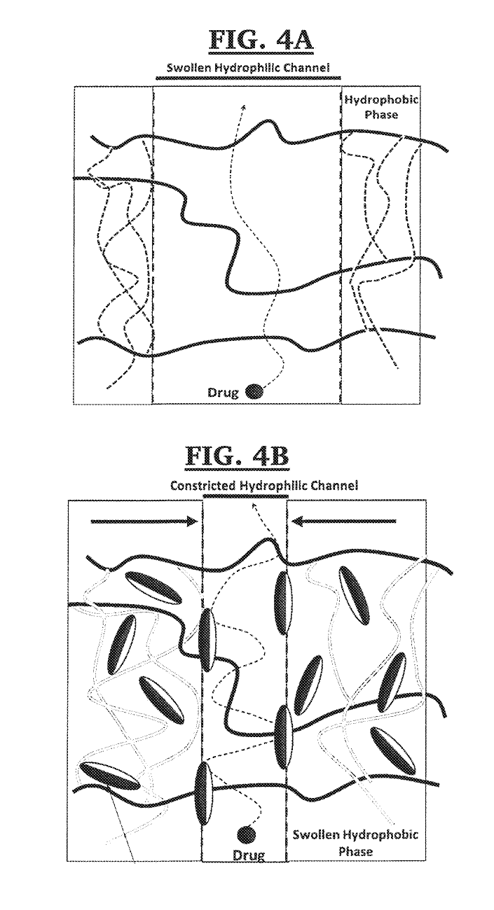

Also, as set forth above, the rate at which a drug diffuses through a β-APCN matrix is also affected by the length of the path it must take through the β-APCN matrix. The more indirect and/or highly circuitous the diffusion pathway through the appropriate pores in the β-APCN, the greater the distance the drug must travel to pass through the matrix and, all other things being equal, the lower the diffusion rate. Conversely, the more direct the diffusion pathway through the appropriate pores in the β-APCN, the shorter the distance the drug must travel to pass through the matrix and, all other things being equal, the higher the diffusion rate. This factor is illustrated, for example, in FIGS. 4A-B. FIG. 4A is a schematic illustration of a drug diffusion pathway of a hydrophilic drug through a β-APCN matrix where a diffusion barrier material is not used and the diffusion pathway is fairly direct through the matrix. FIG. 4B, on the other hand, illustrates a drug diffusion pathway of a hydrophilic drug through a β-APCN matrix having diffusion barrier material, forcing the drug to take a longer, more indirect and circuitous, pathway.

Moreover, vitamin E is a hydrophobic liquid and, as it is incorporated into the β-APCN it is formed into the outer β-APCN layer, it is likely to swell the hydrophobic (PDMS) phase first. It should be appreciated that in β-APCNs, both phases are co-continuous and share a huge interfacial area. Given this morphology, the swelling of the hydrophobic phase will contract the hydrophilic channels available for diffusion (slowing down drug release rates). (See FIG. 4B) showing a swollen hydrophobic phase and contracted hydrophobic channels). Furthermore, by increasing the vitamin E content we can reach its solubility limit in the hydrophobic phase. Further, any excess vitamin E will tend to collect at the hydrophilic/hydrophobic interface and in nano aggregates in the hydrophilic-water-swollen channels (further reducing drug release rates).

As set forth above, the middle layer of the three layer drug delivery device of the present invention will contain a drug or other substance to be delivered into a bodily fluid and/or wound of a patient. The drugs or other substance to be delivered into a bodily fluid and/or wound of a patient using the three layer drug delivery device of the present invention are not particularly limited provided that their dimensions do not exceed that of the channels available for diffusion and they can be loaded into the middle β-APCN layer. Suitable drugs or other substances to be delivered into a bodily fluid and/or wound of a patient may include anti-biotics, anti-microbials, antifungals, pain medications, and steroids. In one or more embodiments, the drug or other substance to be delivered into a bodily fluid and/or wound of a patient may include, without limitation moxifloxacin hydrochloride, dexamethasone, levofloxacin, chlorhexidine, lidocaine, bupivacaine, tetracaine, dyclosporine A, timolol, dexamethasone 21-disodium phosphate, fluconazole, ofloxacin, or combinations thereof.

The drug loading in the middle β-APCN layer of the three layer drug delivery device of the present invention will, of course, depend upon the particular application, and may also be limited by the size of the drug and the size and extent of the pores/channels in the β-APCN layers. The saturation concentration of the drug in the β-APCN will vary with the particular drug being used. In general, however, the drug loading in the middle β-APCN layer of the three layer drug delivery device of the present invention is from about 0.001% by weight to about 1% by weight. In some embodiments, the drug loading may be 1 weight % or more, in other 5 weight % or more, in other embodiments 10 weight % or more, in other embodiments 20 weight % or more, in other embodiments, 30 weight % or more, in other embodiments, 40 weight % or more and, in other embodiments, 50 weight % or more. In some embodiments, the drug loading may be 50 weight % or less, in other embodiments 40 weight % or less, in other embodiments 30 weight % or less, in other embodiments 20 weight % or less, in other embodiments 10 weight % or less, in other embodiments 5 weight % or less, and in other embodiments 1 wt % or less. The maximum drug loading will strictly be limited by the maximum drug solubility in water, which can vary greatly, for example Moxifloxacin Hydrochloride (0.168 mg/mL), Latanoprost (8 mg/mL).

As set forth above, the two outside β-APCN layers of the three layer drug delivery device of the present invention contain diffusional barrier material that retards diffusion through these layers. The diffusional barrier material is not particularly limited and may include many hydrophobic liquids or anisotropic nanoparticles, provided that these materials are biocompatible, non-toxic, and have a relatively small particle size (<40 nm), a low/non-water solubility, good optical clarity (for contact lense embodiments in particular) and a relatively low modulus (in the case of nanoparticles they must not be hard enough to cause mechanical damage to the cornea). Suitable barrier materials may include, but are not the limited to, Vitamin-E (α-tocopherol), nanoclay, or nanoparticles.

The amount of diffusional barrier material present in the two outside β-APCN layers will depend upon the particular system, but will generally be from about 5 weight percent to about 20 weight percent. In one or more embodiments, the amount of diffusional barrier material in the two outside β-APCN layers of the three layer drug delivery device of the present invention will be 3 weight percent, or more, in other embodiments, 6 weight percent or more, in other embodiments, 7 weight percent or more, in other embodiments, 8 weight percent or more, in other embodiments, 10 weight percent or more, and in other embodiments, 12 weight percent or more. In one or more embodiments, the amount of diffusional barrier material in the two outside β-APCN layers of the three layer drug delivery device of the present invention will be 23 weight percent or less, in other embodiments, 19 weight percent, or less, in other embodiments, 18 weight percent, or less, in other embodiments, 17 weight percent, or less, in other embodiments, 15 weight percent, or less, and in other embodiments, 13 weight percent, or less.

In one or more embodiments, the three layer drug delivery device of the present invention may be constructed as follows. First, films of β-APCN, as described above, are prepared having a controlled thickness. The films may be prepared by any appropriate method known in the art for that purpose including, without limitation, blade casting, flow coating, or spin coating. The thickness of these β-APCN films will depend on the particular application and is not particularly limited. In one or more embodiment, the β-APCN films had a final thickness of from about 80 μm to about 200 μm. In some embodiments, the β-APCN films have a final thickness of 85 μm or more, in other embodiments, about 90 μm or more, in other embodiments, about 95 μm or more, in other embodiments, about 100 μm or more, in other embodiments, about 110 μm or more, in other embodiments, about 120 μm or more, and in other embodiments, about 130 μm or more. In some embodiments, the β-APCN films have a final thickness of 190 μm or less, in other embodiments, about 180 μm or less, in other embodiments, about 170 μm or less, in other embodiments, about 160 μm or less, in other embodiments, about 150 μm or less, in other embodiments, about 140 μm or less, and in other embodiments, about 130 μm or less. In some of these embodiments, the films had a final thickness of about 80 μm. In one or more embodiments, the films forming the three layer drug delivery device of the present invention may have different thicknesses.

The films are then cured under vacuum, preferably in a in a vacuum oven. As will be appreciated by those of skill in the art, the curing time will depend upon the oven temperature and the thickness of the films. The curing process allows time for all of the crosslinks to form and heat, if used, will reduce the curing time. As will be appreciated, while the curing temperature is not particularly limited, it must not exceed the T_{d} of the β-APCN. The curing temperature will ordinarily be from about room temperature to about 90° C. In one or more embodiments, the films are cured in a vacuum oven for a period of from about 21 hours to about 24 hours at a temperature of from about 65° C. to about 75° C. In some other embodiments, the films are cured in a vacuum oven at room temperature for a period of about one week. As will be appreciated by of skill in the art, the β-APCN is fully cured when it is no longer tacky or sticky and no extractables are seen after successive solvent treatments. In some embodiments, the films may be cured in a vacuum oven for a period of 24 hours at a temperature of about 70° C.

Next, the drug being delivered to the patient is loaded by any suitable means into the β-APCN film which is to become the middle β-APCN layer of the three layer drug delivery device of various embodiments of the present invention. In one or more embodiment, the drug is first dissolved in a suitable biocompatable solvent or solution appropriate for particular drug being used. As will be appreciated by those of skill in the art, the biocompatable solvent or solution must be non-toxic, non-reactive with the drug being delivered, and compatible with the bodily fluid of the patient into which the drug is to be distributed. In one or more embodiment, the drug being delivered is hydrophilic and is dissolved in a phosphate-buffered saline solution (PBS). Other biocompatible solvents or solutions for hydrophilic drugs may include, without limitation, water, saline solutions, ethanol, N-methyl-2-pyrolidone, or combinations thereof.

Due to the amphiphilic nature of these β-APCNs, hydrophobic drugs can also be used with embodiments of the present invention. In one or more of these embodiments, hydrophobic drugs may be loaded into the continuous hydrophobic-PDMS channels of the inner β-APCN layer, as outlined above for hydrophilic drugs. Suitable biocompatible solvents or solutions for hydrophobic drugs will, of course, depend upon the drug being used but may include biocompatible solvents for hydrophobic materials such as decane. It should be appreciated, however, that the outward diffusion of such drugs into the body of a patient will be much slower than that of hydrophilic drugs due to their low water solubility. Alternatively, microemulsions of these hydrophobic drugs (with a particle size small enough <40 nm) in a biocompatable hydrophilic solvent or solution may be prepared using any suitable method known in the art for that purpose, and then loaded into the continuous hydrophilic-PDMS channels of the inner β-APCN layer, as outlined above for hydrophilic drugs.

In some of these embodiments, β-APCN film that is to become the middle β-APCN layer of the three layer drug delivery device of various embodiments of the present invention is first dried and then placed in the biocompatible solution containing the drug to be delivered and allowed to swell. As the β-APCN film swells, the drug is pulled into the interstitial pores within the film, thereby drug loading the film. The β-APCN film is allowed to swell in the biocompatible solution containing the drug until a desired amount of the drug has been loaded into the film. In one or more embodiment, the β-APCN film is allowed to swell for from 1 hour to 6 days. In one or more embodiment, the β-APCN film is allowed to swell for from 2 days to 5 days. As set forth above, the β-APCN materials forming these layers have both hydrophilic and hydrophobic pores and will swell when placed in hydrophilic and/or hydrophobic solutions. Accordingly, both hydrophilic and hydrophobic drugs may be loaded into the middle β-APCN layer in this manner. Further, as set forth above, the β-APCN materials must have pores of sufficient size to accommodate the particular drug being used. In one or more embodiments, the β-APCN materials will have pore sizes as described above. In one or more embodiments, the β-APCN materials will have pore sizes as large as 50 nm.

In some other embodiments, the drug may be loaded into the into the inner β-APCN layer during formation of the layer by adding it into reaction mixture during formation of the β-APCN and before the final crosslinking step. As will be apparent, care must be taken in these embodiments to prevent damaging or denaturing the drug during formation of the β-APCN.

As set forth above, the two films that will become the outer β-APCN layers of the three layer β-APCN based drug delivery device of various embodiments of the present invention contain a diffusional barrier material, such as vitamin-E. The diffusional barrier material may be introduced into the outer β-APCN layers in any suitable manner known in the art. In one or more embodiment, a measured amount of the blocking material maybe stirred into reaction mixture during formation of the β-APCN and before the final crosslinking step. In some embodiments, the diffusional barrier material (vitamin E) loading was done by weighting and adding a specific amount of a-tocopherol to the reaction mixture under strong stirring for 20 minutes.

Alternatively, diffusional barrier material may be loaded into the outer β-APCN layers by allowing the diffusional barrier material to diffuse into the same manner as the hydrophilic drugs described above. In these embodiments, a dried cured β-APCN film is placed in a solution containing the solution diffusional barrier material and allowed to swell, thereby pulling the diffusional barrier material into the β-APCN film. In one or more of these embodiments, the β-APCN film is placed in an ethanol/Vitamin E solution and allowed to swell.

Finally, the three films are joined to create the three layer β-APCN based drug delivery device of various embodiments of the present invention. The films may be joined by any suitable means known in the art provided that the process does not introduce an impermeable barrier between the β-APCN layers when they are joined or flush some, or all, of the drug out of the middle β-APCN layer during the process. Suitable methods for joining the three β-APCN layers include, without limitation, hot-pressing, adhesives, such as cyanoacrylates (see “Cyanoacrylate Adhesives in Surgical Applications” Petrie, Edwards M., No 3/August 2014, PP 253-310 (58), Scrivener Publishing, the disclosure of which is incorporated herein by reference in its entirety) or by applying a small amount of unreacted β-APCN, which is then cured to adhere the β-APCN layers together. In one or more embodiments, the three β-APCN films are stacked with the middle, drug loaded β-APCN film placed between the two outer diffusional barrier material carrying β-APCN films, and joined by hot-pressing them together in a hydraulic press at a pressure of from about 200 Psi to about 1000 Psi and a temperature of from about 100° C. to about 120° C. In some embodiments, the three β-APCN films are joined by hot-pressing them together in a hydraulic press at 1000 Psi and 100° C.

As set forth above, in one or more embodiments, the three layer β-APCN based drug delivery device of the present invention provides zero-order drug release kinetics at the β-APCN/bodily fluid interface. When the device is placed into the bodily fluid of a patient, the drug will begin to diffuse from its initial location within the middle β-APCN toward the interface between the middle β-APCN layer and the outer β-APCN layers. Because the drug diffuses faster through the middle β-APCN layer than the outer β-APCN layers containing the diffusional barrier material, the middle β-APCN layer acts as a reservoir for the drug, which diffuses through the outer layer at a constant rate that is substantially independent of the concentration of the drug in the middle layer. The drug release kinetics are zero-order since neither the location nor the concentration of the drug in the middle β-APCN layer affect the rate at which the drug is released from the three layer β-APCN based drug delivery device of the present invention into the bodily fluid of the patient.

As will be discussed in more detail in the Experimental section below, the three layer β-APCN based drug delivery device of the present invention may be configured for use as a therapeutic contact lens. Like a conventional contact lens, these lenses are placed in the eye of the patient, between the eye (cornea) and the inside surface of the eyelid. In these embodiments, the β-APCN selected must be translucent over the visual spectrum and have good oxygen permeability. As will be discussed below, it has been found that β-APCNs comprising a crosslinked percolating hydrophilic poly(N,N-dimethylacrylamide) (PDMAAm) and hydrophobic polydimethylsiloxane (PDMS) networks allow for high oxygen permeation and improved mechanical properties and may be optimized in order to maximize oxygen permeation.

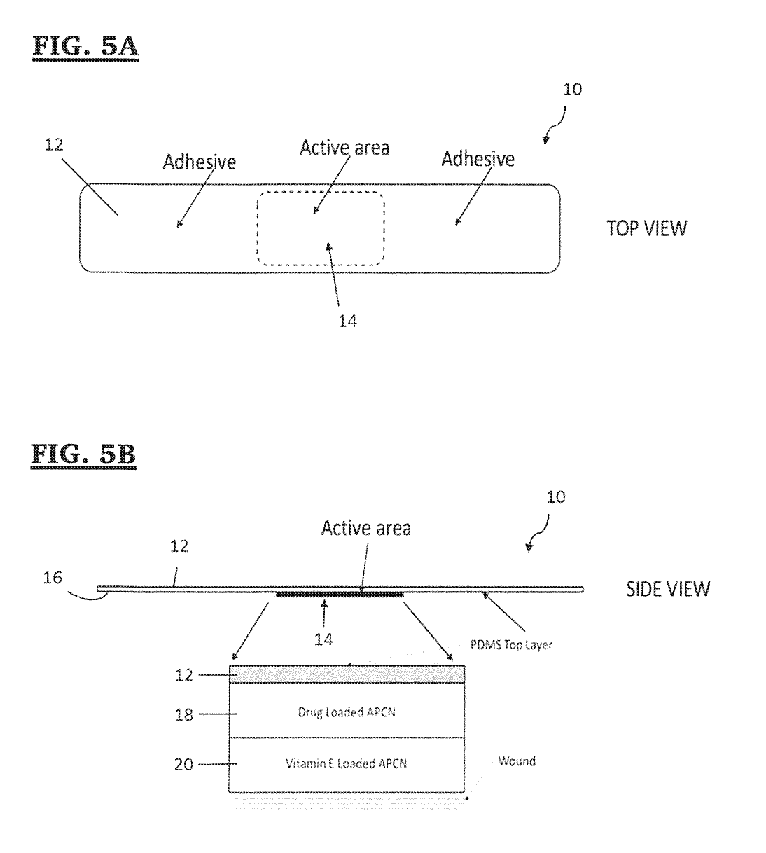

In some other embodiments, the three layer β-APCN based drug delivery device of the present invention may be configured for use as a wound dressing as shown in FIGS. 5A-B. In these embodiments, one of the two outer layers is blocked (is impermeable to the drug) so that all of the drug is released through the other outer layer. In these embodiments, one of the outer layers may be blocked by any means known in the art including, without limitation, polydimethylsiloxane (PDMS), poly(lactide-co-glycolide) (PLGA), polylactic acid (PLA), polyacrylates or any other biocompatible polymer that does not swell in water. In these embodiments, the three layer β-APCN based drug delivery device of the present invention is placed over a wound with the blocked outer layer facing away from the patient's wound. (See FIG. 5)

In the Embodiments shown in FIGS. 5A, the wound dressing 10 includes a PDMS top layer 12 that is impermeable to the drug being delivered and has an active area 14 containing the drug to be delivered and an adhesive underside 16 that is removably secured to the area around the wound by an adhesive that holds wound dressing 10 in place with the active area 14 over the wound. FIG. 5B shows a side view of wound dressing 10 and an enlarged view of the active area 14 showing the three layer structure comprising a the drug impermeable PDMS top layer 12, the drug loaded APCN layer 18 and the vitamin E loaded APCN 20.

### Experimental

The drug delivery system of the present invention was examined for delivery of a topical antibiotic from a therapeutic contact lens made using a specially synthesized three layer bimodal amphiphilic conetwork (β-APCN) with the use of diffusion barriers. Non-Fickian kinetics were achieved via a non-uniform drug concentration distribution within the lens and a significant difference in diffusion coefficients between the middle and outer layers were observed. Special emphasis was placed on the effect of local drug concentration at the lens-fluid interface.

### Bimodal Amphiphilic Co-networks Characterization and Drug Release Profiles.

As set forth above, bimodal amphiphilic conetworks (β-APCN) provide a unique route to integrate contrasting attributes of otherwise immiscible components within a single material. This characteristic allows them to exhibit unique properties and makes them exceptional materials for therapeutic contact lenses. The presence of a co-continuous morphology of percolating hydrophilic poly(N,N-dimethylacrylamide) (PDMAAm) and hydrophobic polydimethylsiloxane (PDMS) networks allows for high oxygen permeation and improved mechanical properties. In this system, the hydrophobic moiety (PDMS) has been carefully chosen in order to maximize oxygen permeation. Additionally, the PDMS phase is bimodal, having a mixture of high and low molecular weight chains, which both greatly enhances the mechanical properties and provides us with an extra control dial of functional properties. The synthesis and characterization of Bimodal Amphiphilic Conetworks was reported before. See, e.g., G. Guzman, T. Nugay, l. Nugay, N. Nugay, J. Kennedy, M. Cakmak, Macromolecules 2015, 48, 6251 and International Published Patent Application No. WO 2014/197699, the disclosures of which are incorporated herein by reference in their entirety. See also, Example 2 below.

Briefly, the synthesis procedure encompasses the free radical terpolymerization of N,N-dimethylacrylamide (DMAAm) with a statistical mixture of telechelic macronomers of low and high molecular weight (1, 2 or 5% HMW-PDMS) PDMS, carrying either -vinylsilyl (-V) or -methacrylate (-MA) terminations (MA-PDMS-V, V-PDMS-V and MA-PDMS-MA). A bimodal amphiphilic graft (bAPG) consisting of PDMAAm main chains carrying -PDMS-V branches is obtained as result. Due to the presence of MA-PDMS-MA chains, the graft is slightly crosslinked and of high molecular weight. Grafts of varying of % HMW-PDMS are then mixed with PHMS-co-PDMS crosslinker in several mole ratios (allyl chain end/hydrosiloxan), and with Karstedt's catalyst in THF. Samples were prepared by blade casting. See, Example 2 below.

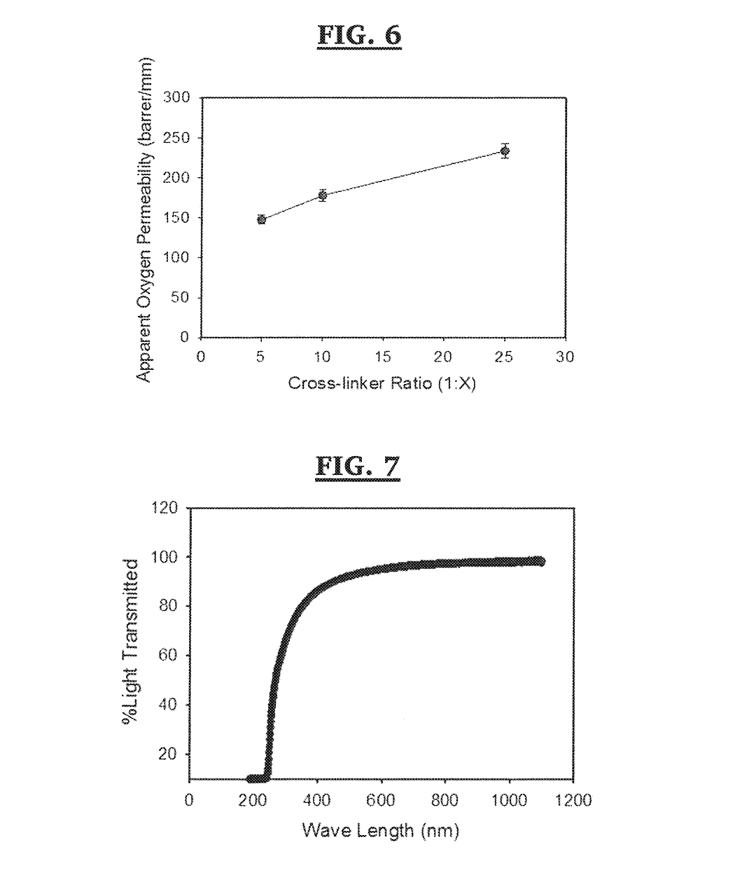

In order to be effective as therapeutic contact lenses, the lenses material must have high oxygen permeability. Apparent oxygen permeability of β-APCNs was determined at 37° C. The instrument used together with specifications and the operational principle were described in detail elsewhere. FIG. 6 presents the apparent oxygen permeability for β-APCN with different crosslinker ratios. Increasing the crosslinker ratio from 1:5 to 1:25 has been found to increases the apparent oxygen permeability of the material from approximately 150 barrer to about 230 barrer. As oxygen permeation occurs mainly through the hydrophobic siloxane phase, an increase in the amount of PHMS-co-PDMS crosslinker likely increases the available domains for oxygen permeation. As a result, β-APCNs based contact lenses of any of the studied compositions would possess more than adequate oxygen permeability for extended wear.

By the presence of a percolating hydrophilic network, the material can be highly swollen in aqueous solution, allowing the loading and subsequent release of molecules of interest like antibiotics. This is carried out by controlling the molecular weight of the hydrophilic segments and cross-linking of the network, which, in turn, controls the diffusion rate of drug and facilitates control of the release profiles. β-APCN materials are optically clear (see transmission values in FIG. 7) and possess the necessary mechanical properties to properly function as therapeutic contact lenses. Drug loading was achieved by soaking β-APCN films in a PBS-moxifloxacin hydrochloride solution. Moxifloxacin was chosen as it is one of the most commonly used antibiotics for eye infections. After loading, samples were transferred into a PBS release solution. At predetermined time intervals, the solution surrounding the sample film was removed, stored for analysis, and replaced with fresh PBS. The collected samples were analyzed by UV-Vis spectroscopy. The tests were carried out in triplicate.

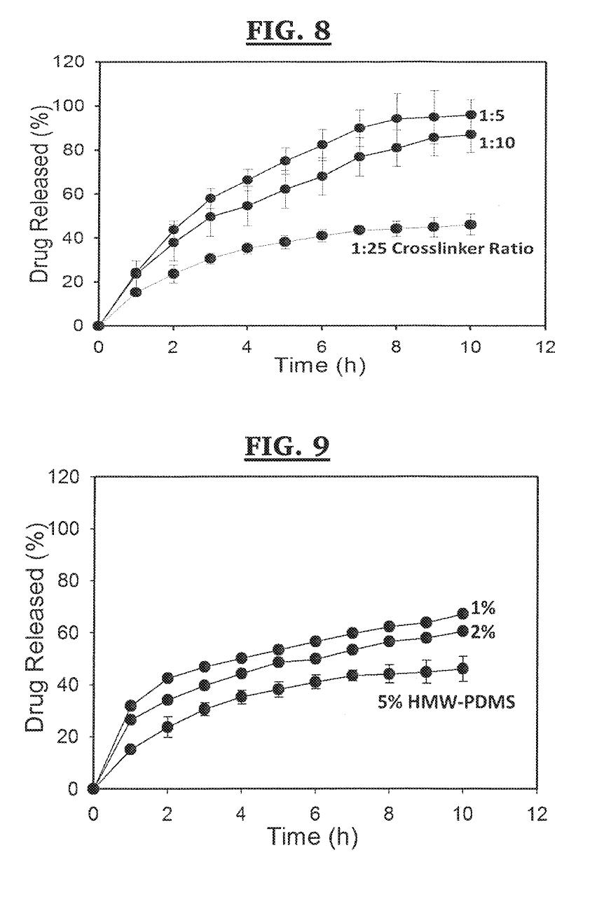

Under these conditions, bimodal amphiphilic conetworks released between ˜35 and 100% of the drug in about ten hours, as shown in FIGS. 8 and 9. Samples with a higher crosslinker ratio presented considerably slower release kinetics. The same effect was observed in samples with a higher percentage of HMW-PDMS, albeit less pronounced. In the swollen state, moxifloxacin hydrochloride permeates only through hydrophilic channels formed by water-swollen PDMAAm domains. The dimensions of these channels are controlled by the molecular weight between crosslinks (M) of the hydrophilic moiety and by morphological thermodynamic/kinetic constrains on the network. M_{c} was calculated by:

$${M}_{c} = \frac{( {M}_{n , PDMAAm} \cdot {M}_{n , MA \text{-} PDMS \text{-} V} \cdot {W}_{PDMAAm} )}{\begin{matrix}( {W}_{MA \text{-} PDMS \text{-} V} \cdot {M}_{n , PDMAAm} ) + \\ ( {M}_{n , MA \text{-} PDMS \text{-} V} \cdot {W}_{PDMAAm} )\end{matrix}} \qquad ( Eq . 3 )$$

where, W_{PDMAAm} and W_{MA-PDMS-V} are the weights of PDMAAm and acrylate end functionalized PDMS, respectively, M_{n,PDMAAm} is the number average molecular weight of PDMAAm determined by GPC, whereas M_{n/MA-PDMS-V} is that of acrylate end functionalized PDMS. With calculations based on experimental data, equation 3 yields an M_{c} of 9915 g/mol, which represents domains large enough to allow fast moxifloxacin permeation. This calculation is based on calculated Mₙ PDMAAm in case of 100% initiator efficiency (AIBN) and some experimental data (Mₙ PDMS, WPDMS and WPDMAAm). This is the best method for calculating approximate value of M_{c}. Another method for M_{n PDMAAm} determination involves performing DMAAm homo- polymerization under the same experimental conditions as the graft copolymerization and then finding the Mₙ by GPC characterization. In M_{c} calculation M_{n PDMAAm,GPC} can be used instead of above mentioned calculated Mₙ.

Cross-links in the hydrophobic phase contract the network and, as anticipated, an increased crosslinker ratio increases drug release time. On the other hand, the molecular weight of the short PDMS chains is below the entanglement molecular weight of PDMS (47,000 g/mol), while that of the long chains is considerably above this value. In this manner, the number of entanglements considerably increases by increasing the content of HMW-PDMS in the network. Some fraction of these entanglements present in the bulk polymer before cross-linking become permanently trapped during network formation and act as additional cross-links, constraining the network and increasing drug release times. Still, the release profile of all β-APCN grades was concentration-dependent, first order “burst” kinetics. At best, a highly cross-linked, high molecular weight β-APCN grade would be able to provide drug for one to two days.

Drug Release from Vitamin E-loaded β-APCNs.

A considerable amount of research has been directed towards using vitamin E-loaded contact lenses for delivery of ophthalmic drugs. The present research is not focused on furthering the understanding of vitamin E loading on the diffusion rate of drugs, but rather employs this technique as an effective and proven method to decrease the diffusion coefficient of hydrophilic drugs through silicon-hydrogel-like materials within a larger context.

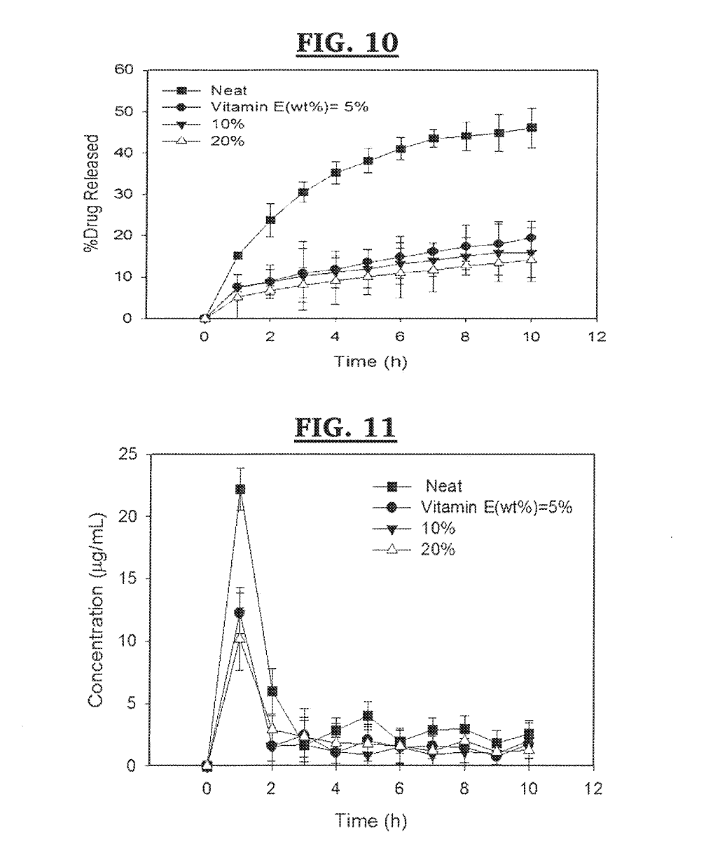

Vitamin E loading was carried out by adding a specific amount of α-tocopherol (5, 10 and 20 wt %) to the reaction mixture before film casting. The drug was loaded into the vitamin E loaded films using the same procedure employed before except for increasing the loading time to 4 days. Release experiments were performed following the same procedure used for neat samples. FIGS. 10 and 11 present moxifloxacin hydrochloride release profiles (FIG. 10 shows % drug released; FIG. 11 shows concentration profiles) for β-APCN (2% HMW-PDS, 1:25 crosslinker ratio) with vitamin E loading levels ranging from about 5 to 20%. Vitamin E loaded samples exhibited considerably slower release kinetics compared to neat samples. At 5% vitamin E loading, only about 19% of the drug was released at 10 hours, which is a significant improvement when compared to the about 49% of the neat sample. At 10 and 20% vitamin E loading, the released percentage is further reduced to about 15 and about 14% respectively. Interestingly, a significant difference in release times is observed between the 5% vitamin E loaded and neat samples, but the effect saturates at higher vitamin E concentrations (10 & 20%). Vitamin E loaded samples still present an initial burst in concentration at early release times. The burst is considerably lower in the vitamin E loaded samples as compared to the neat samples, but the release is still diffusion controlled. These results show us that by using a diffusional barrier one is able to slow the kinetics of release without changing the mode of transport. As long as the release process is diffusion limited and the concentration at the lens-fluid interface is high, the release rate will be very fast at early times and a burst will be observed.

Vitamin E has a refractive index of n=1.506. A suspension of vitamin E aggregates in water (n˜1.33) would cause light scattering making the lens opaque, unless the particle size is in the nanometer scale. As the films loaded with vitamin E remained transparent, the assumption so far has been that the sizes of the aggregates are smaller than the wavelength of the visible light (<400 nm). The question remains of why the aggregates should have such a small particle size. Vitamin E has a very high viscosity compared to water (which promotes the formation of large droplets) and even when using multicomponent mixtures and surfactants, it is non-trivial to achieve transparent vitamin E emulsions.

Due to their co-continuous nature, it is possible to simultaneously swell the hydrophilic and hydrophobic phases of Amphiphilic Conetworks with different solvents. At the same time PDMS is able to swell and permeate a-tocopherol. It is safe to assume then, that the loaded vitamin E will first swell the hydrophobic phase, constricting the hydrophobic channels and increasing drug release times, (see FIG. 4B) up to the saturation point. Beyond this point vitamin E would be first located at the hydrophobic-hydrophilic interface and later in the hydrophilic phase itself with increasing concentration. As mentioned before, the size of hydrophilic domains depends on the molecular weight between crosslinks (M_{c}) and while it can vary significantly, it is unlikely to reach ˜400 nm. In our case, with M_{c}˜10,000 g/mol, the size of the hydrophilic channels is much smaller than any visible wavelength. In this manner, the vitamin E loaded silicon-hydrogel lens remains transparent, as the size of the vitamin E aggregates is likely kept small by the size of the hydrophilic channels themselves.

### Triple Layer System and Low Local Interfacial Concentration Hypothesis

When analyzing mass transfer phenomenon in therapeutic contact lenses, the transport is usually modeled by the following equation:

$$\frac{\partial C}{\partial t} = D \frac{{\partial }^{2} c}{\partial {y}^{2}} \qquad ( Eq . 2 )$$

where C is the drug concentration in the lens, D is the effective diffusivity and y and t denote the transverse coordinate and time, respectively. It is assumed that the surrounding fluid volume is much larger than lens volume, that the drug is uniformly distributed in the lens and the solubility of the drug is very high in the surrounding media. When setting the proper boundary conditions under perfect sink, and solving equation 3, it is possible to obtain a time function for both the concentration profile in the lens and the concentration in the release media. The concentration in the release media is found to be a linear function of the initial concentration and a square root function of time and the diffusion coefficient, C(D^{0.5},t^{0.5}, Cᵢ). The result is similar to the fickian case of the Higuchi equation (Eq. 1).

In order to achieve constant-rate drug delivery we have selected a system in which a non-uniform drug profile and non-uniform diffusivity distribution within the lens is able to maintain, at all times, a low local concentration at the boundary between the lens and the release media. This is particularly important at just after administration when the drug concentration in the lens is high.

The proposed lens is composed by a three-layer system: The center layer is composed by a β-APCN matrix and contains a high drug loading. Two outer layers which are also β- APCN-based, contain no-drug and are instead loaded with vitamin E. As we have demonstrated, the vitamin E loaded layer will possess a considerably smaller diffusion coefficient. FIG. 3A presents a simple illustration. The large and rapidly retrievable reservoir of drug in the center layer must first slowly diffuse through the outer layers. Once the drug concentration profile reaches the lens-fluid interface, the drug is quickly removed under sink conditions. In this manner, the concentration at the lens-tear layer interface is low when the lens is first placed in the eye, thus preventing a “burst” release.

### Transient Diffusion Model in a Three-Layer Structure

In this section, a simple model is used to display how adding two layers with small values of diffusion coefficient on both sides of a layer loaded with drug can slow down the diffusion process and approach zero order kinetics. FIGS. 3A-B provide a schematic representation of one dimensional diffusion in a three-layer structure where the diffusion coefficient in the lateral layers is different from that in the middle layer. The governing equations, initial and boundary conditions are also shown in FIG. 3B.

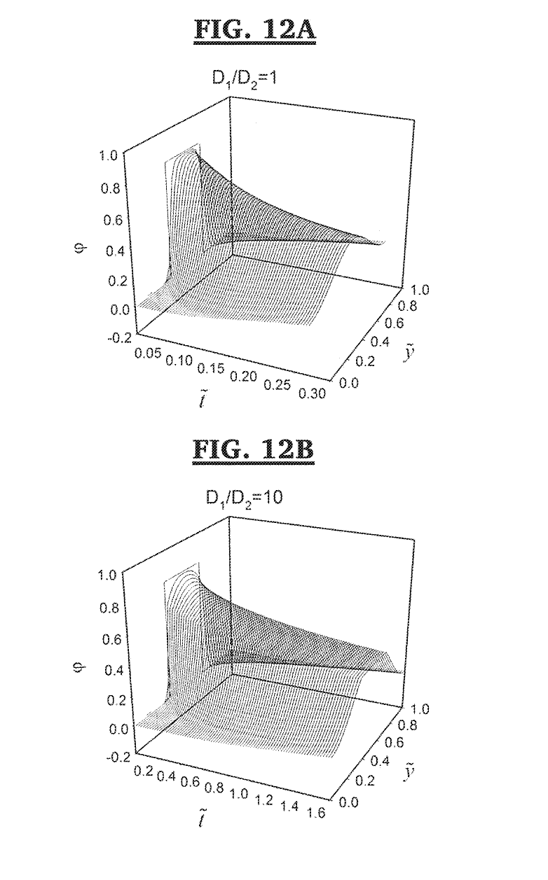

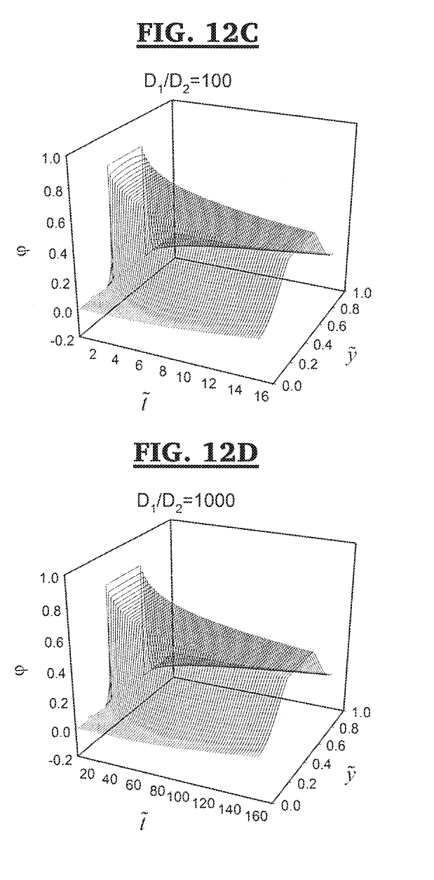

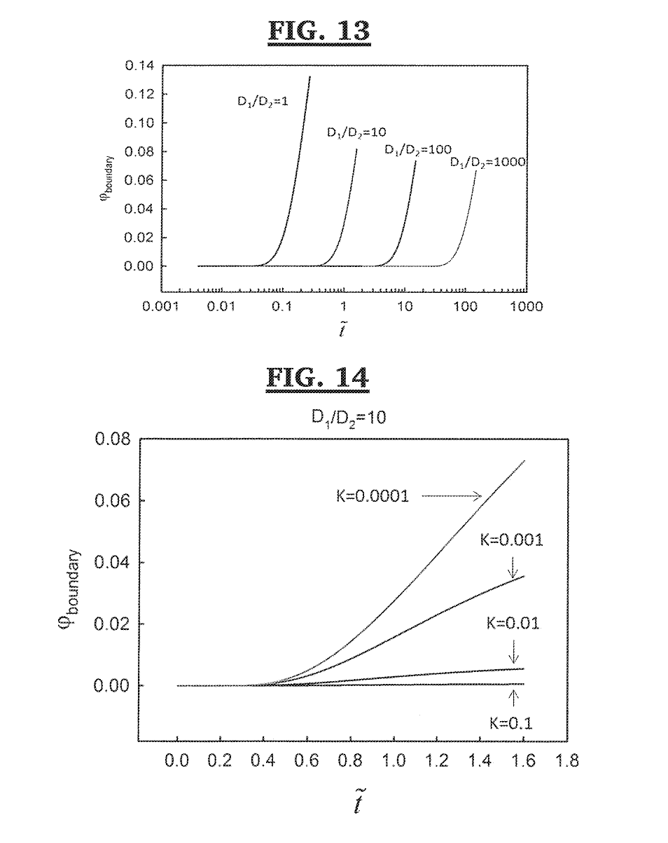

Only the middle layer contains drug, which is uniformly distributed with an arbitrary initial concentration φ₀. The boundary conditions at the lateral sides were formulated so that the outward flux is a linear function of concentration with the slope of K that can be related to the permeability of the environment. The governing equations and associated initial and boundary conditions were first non-dimensionalized and then the problem was solved numerically using an explicit finite difference scheme. As will be shown later, the ratio of diffusion coefficients D₁/D₂ plays an important role in determining the kinetics of diffusion. D₁, is the diffusion coefficient in the middle layer loaded with the drug. The initial non-dimensional concentration of the drug in the middle layer was considered φ₀=0.8, arbitrarily. D₂ is the diffusion coefficient in two other layers attached to the middle layer. In order to solve the problem, the thickness of layers was assumed to be the same and the effect D₁/D₂ on the diffusion kinetics was studied by setting D₁ to 10⁻⁶ m²/s and considering different values for D₂, 10⁻⁶, 10⁻⁷, 10⁻⁸, and 10⁻⁹ m²/s. K was initially (and conservatively) assumed to be equal to D₁. Simulation results are shown until the concentration of drug in the center of the middle layer reaches 50% of its initial concentration. FIGS. 12A-B shows the concentration profiles as a function of dimensionless time \tilde{t} and dimensionless thickness of the three-layer structure \tilde{y}. As one can see, by increasing D₁/.D₂ , which indicates slower diffusion in lateral layers as compared to the middle layer, the initial concentration of the drug more slowly reaches the 50% of its initial value. For the case where diffusion coefficient in three layers are the same, D₁/D₂=1, the drug is quickly releases from the structure. This can be observed better by looking at the values of concentration at the boundary shown in FIG. 13. In case of D₁/D₂=1, the concentration at the boundary quickly jumps to high values, however, by decreasing D₂, the concentration at the boundary is lower and it will be kept low for longer times. It should be noted that since the three-layer structure is symmetric, the concentration profiles are symmetric as shown in FIGS. 12A-D, and concentration at both boundaries are the same.

As mentioned earlier, for computations shown above, K was assumed to be equal to D₁. The value of K plays an important role in determining concentration at the boundaries. To study the effect of K on the diffusion kinetics, we consider the case where D₁/D₂=10 and performed computations for different values of K. FIG. 14 shows the concentration at the boundary for different values of K. By decreasing K, the concentration at the boundary increases. It shows that when the permeability of the environment is high the concentration at the boundary remains low. The results of this simple model are of high, albeit qualitative, significance. It shows us that having a triple layer system in which the outer layers possess a smaller diffusion coefficient compared to the drug-loaded middle layer, can effectively keep the local concentration at the boundary low and quasi-constant. In this manner, the release rate, which is concentration dependent within each layer, can be kept close to constant for the multilayer system, and the initial “burst” can be avoided. Moreover, time scales for diffusion in the tear layer and the drug absorption kinetics can be safely assumed to be significantly faster than that for drug diffusion across the lenses. Consequently, the value of K in-vivo is larger than D₁, and much larger than D₂, further keeping the concentration at the boundary low.

Release Profiles from Triple Layer System

In order to corroborate the results of the model, multi-layer samples were prepared by hot-pressing three fully cured films. A film of moxifloxacin hydrochloride-loaded neat β-APCN polymer (2% HMW-PDMS, 1:25 crosslinker ratio) was sandwiched in between two vitamin E loaded (10%) β-APCN films that contained no drug. The loading and release procedures employed were the same as with vitamin E loaded single layer samples. FIG. 2 presents concentration profiles for neat β-APCN, vitamin E loaded (10%), and triple layer samples. Remarkably, triple layer samples present a constant-rate drug delivery with no initial burst, while keeping an overall higher drug concentration than the neat and vitamin E loaded single-layer samples.

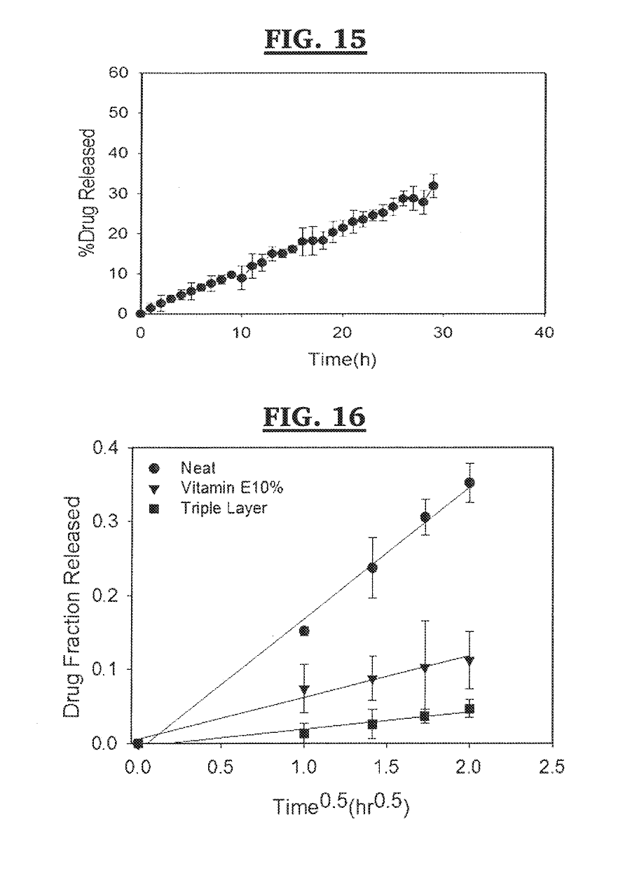

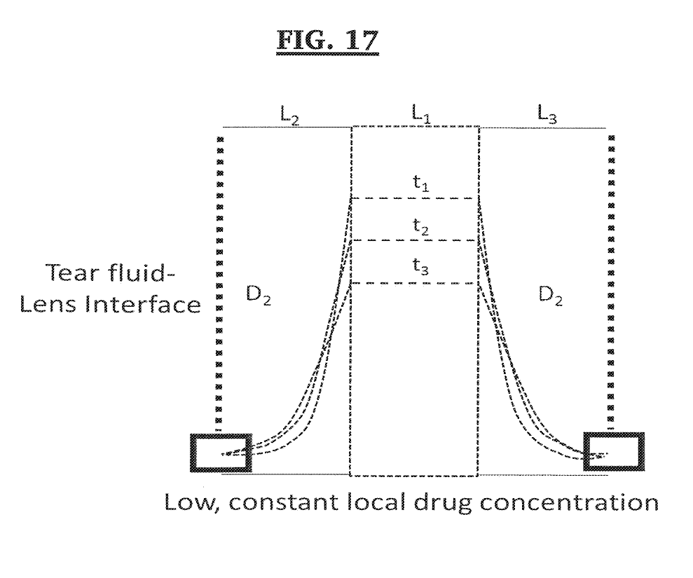

The lack of a burst release in triple layer samples is accompanied by a linear evolution of the drug fraction released, as seen in FIG. 15. The linear profile is indicative of zero order release kinetics, as the exponent in the Higuchi equation approaches unity. This characteristic can be better seen in FIG. 16 where the release profiles for the neat, vitamin E loaded and triple layer samples are plotted as a function of square root of time. The lines in the figure are the best fit straight line to short-time release data. Both neat and vitamin E loaded samples show a good fit with R²>0.99, indicating Fickian transport, as the exponent in the Higuchi equation (Eq. 1) equates 0.5. The triple layer sample on the other hand has a poor correlation indicating an anomalous (non-fickian) diffusion mode. As predicted by the model, the local concentration at the lens-fluid interface is kept low by a combination of non-uniform drug concentration distribution within the lens and the difference in diffusion constants between the middle and outer layers. FIG. 17 illustrates these matters. It is worth noting that even when the loading Moxiflixacin solution concentration was only about 50 μg/mL (compared to >5000 μg/mL in Eyedrops) the constant concentration release achieved by the triple layer samples is above the minimum inhibitory (MIC₉₀) concentration of Moxifloxacin Hydrochloride for most of the common bacteria strains.

### Conclusions

In these experiments, bimodal Amphiphilic Conetworks were presented as contact lenses matrices, able to hold and release Moxifloxacin Hydrochloride at different rates, while simultaneously fulfilling all other necessary requirements for extended wear contact lenses most importantly high oxygen diffusion characteristics. It was also shown that the use of diffusional barriers, like vitamin E, alone is not enough to achieve zero-order release kinetics, as even when diffusion rates were considerably slowed, the mode of transport (fickian) remains concentration-dependent and a burst release is observed. The use of a multilayer system in which the middle layer contains the drug and the outer layers contain diffusional barriers was shown, theoretically and experimentally, to be able to keep a comparatively low local concentration at the interface between the lens and the surrounding fluid. Constant-rate drug delivery was achieved without an initial burst and at rates consistent with several days of antibiotic release above the therapeutic level. These findings expand the fundamental understanding of therapeutic contact lenses, and the results presented here can be widely applicable to other therapeutic contact lenses systems as well as drug delivery systems in general.

### Examples

The following examples are offered to more fully illustrate the invention, but are not to be construed as limiting the scope thereof. Further, while some of examples may include conclusions about the way the invention may function, the inventor do not intend to be bound by those conclusions, but put them forth only as possible explanations. Moreover, unless noted by use of past tense, presentation of an example does not imply that an experiment or procedure was, or was not, conducted, or that results were, or were not actually obtained. Efforts have been made to ensure accuracy with respect to numbers used (e.g., amounts, temperature), but some experimental errors and deviations may be present. Unless indicated otherwise, parts are parts by weight, molecular weight is weight average molecular weight, temperature is in degrees Centigrade, and pressure is at or near atmospheric.

### Example 1

### Materials

The synthesis and characterization of Bimodal Amphiphilic Conetworks was reported before. See e.g., G. Guzman, T. Nugay, l. Nugay, N. Nugay, J. Kennedy, M. Cakmak, Macromolecules 2015, 48, 6251 and International Published Patent Application No. WO 2014/197699, the disclosures of which are incorporated herein by reference in their entirety. Polyhydrosiloxane-PDMS copolymer (PHMS-co-PDMS) containing 30% PHMS, and Karstedt's catalyst (3% Pt in xylene, low color) were purchased from Gelest and used without further purification. Tetrahydrofuran (THF) and a-tocopherol were obtained from Sigma Aldrich. Moxifloxacin Hydrochloride was obtained in its commercial eyedrop form, Vigamox™.

### Sample Preparation

Grafts of 1, 2, and 5% HMW-PDMS (0.9 g) were mixed with crosslinker in three mole ratios (allyl chain end/hydrosiloxane=1:5, 1:10 and 1:25), and Karstedt's catalyst (25 uL) in THF (8 mL). The bAPGs were mixed with PHMS-co-PDMS crosslinker by strong stirring in THF for 10 min. Films of controlled thicknesses (˜80 um final thickness) were prepared by blade casting and subsequently cured in a vacuum oven for 24 hours at 70° C. Vitamin E loading was done by weighting and adding a specific amount of a-Tocopherol (2, 5, 10 and 20 wt %) to the reaction mixture under strong stirring for 20 minutes. Multi-layer samples were prepared by hot-pressing three fully cured 2×2cm films in a hydraulic press at 1000 Psi and 100° C.

### Oxygen Permeability

Apparent oxygen permeability of β-APCN grades with different crosslinker ratios was determined at 37° C. The instrument used together with specifications and the operational principle are described in G. Erdodi, J. P. Kennedy, J. Polym. Sci. Part A Polym. Chem. 2005, 43, 3491, the disclosure of which is incorporated herein by reference in its entirety. Blade casted samples of 10 cm×10 cm×0.1 mm were used.

### Drug Loading and Release

Drug loading was achieved by soaking previously dried 2×2cm films in 5 mL of PBS-Moxifloxacin Hydrochloride solution of 50 ug/mL for 2 days, at room temperature. After loading, the lenses were taken out from the solutions and blotted with absorbent paper before being transferred into the release solution The drug was loaded into the vitamin E loaded films and the Multiple-layer films using the same procedure except for increasing the loading time to 4 days. The drug-loaded films were immersed in 5-mL PBS, which can be considered infinite sink conditions. The samples were kept in a G24 Environmental incubator shaker from New Brunswick Scientific, mixed at 100 rpm and at 37° C. At predetermined time intervals, the solution surrounding the sample film would be removed, stored for analysis, and replaced with 5-mL of fresh PBS. The collected samples were placed in a quartz cuvette, and analyzed in a UV-Vis spectrophotometer (Beckmam DU-70). Moxifloxacin hydrochloride concentration was determined by following the absorbance of peaks at 204 and 289 nm and comparing with calibration curves. The tests were carried out in triplicate and the results are shown in FIGS. 2, 8, 9, 10, 11, 15, and 16.

### Example 2

### Calculation of Molecular Weight between Crosslinks (M_{c}) Based on Experimental Data

The molecular weight between crosslinks (M_{c}) for bimodal co-networks of crosslinked poly(N,N-dimethylacrylamide) (PDMAAm) and polydimethylsiloxane (PDMS), as described in Example 1 above, was calculated based on experimental data as follows.

In a first step, the number of PDMAAm chains was calculated from the number of moles of DMAAm used to form the β-APCN. To do this, the number average molecular weight (Mₙ) of the PDMAAm was measured by gel permeation chromatography (GPC) and recorded and the initial weight of N,N-dimethylacrylamide (DMAAm) (3.57 g) was likewise measured and recorded. The initial weight of DMAAm then divided by the measured Mₙ to provide the number of moles of PDMAAm, which was reasonably assumed to be the number of PDMAAm chains in moles.

Alternatively, the number average molecular weight of the PDMAAm chains could have been calculated from the initial weight of N,N-dimethylacrylamide (DMAAm) (3.57 g), assuming a 1:1 molar relationship between the PDMAAm and the radical initiator. Here, 0.032 mmol of azobisisobutyronitrile (AIBN) radical initiator was used, providing 0.064 mmol of radicals and having an efficiency (f) of 1. Assuming f=1, the reaction will produce 0.064 mmol (0.064×10⁻³ moles) of PDMAAm chains in the polymer. Dividing the weight of DMAAm (3.57 g) by the number of moles (0.064×10⁻³ moles) provides a minimum molecular weight of PDMAAm of 55781 g/mol. Next, the number of acrylate end functionalized PDMS (PDMS-Ac) chains may be calculated by dividing the weight of PDMS-Ac used (3g) by the Mₙ of these PDMS-Ac macromolecules as measured by ¹H NMR (value of M_{n NMR V-PDMS-V (}11600 g/mol) +value of SiH-MA (232 g/mol) =Mₙ of V-PDMS-MA (11832 g/mol)). Here, the number moles of PDMS-Ac chains will be 3 g/11832 g/mol or 2.54×10⁻⁴ mol.

The second step is to calculate the number of crosslinks or other branches per PDMAAm chain. Given the information provided above, the number of PDMS-Ac branches per PDMAAm chain (X) may be calculated as follows:

$$\begin{matrix}X = \frac{moles of PDMS \text{-} Ac chain}{moles of PDMAAm chain} \\ or & Eq . 4 \\ X = \frac{Weight of PDMS \text{-} Ac / {M}_{n} PDMS \text{-} Ac ( NMR )}{Weight of DMAAm / {M}_{n} PDMAAm ( GPC )} & Eq . 5\end{matrix}$$

Finally, the molecular weight between crosslinks (M_{c}) was calculated from the number of PDMS-Ac branches per PDMAAm chain (X). As will be appreciated, X PDMS-Ac branches on a PDMAAm chain effectively divides the PDMAAm chain into X+1 segments. Accordingly, the molecular weight between crosslinks (M_{c}) will be the molecular weight of the PDMAAm chain divided by the number of segments (X+1).

$$\begin{matrix}{M}_{c} = \frac{{M}_{n} PDMAAm ( GPC )}{X + 1} \\ or & Eq . 6 \\ {M}_{c} = \frac{{M}_{n} PDMAAm ( Calculated ) / f}{\frac{X}{f} + 1} & Eq . 7\end{matrix}$$

was also be calculated from the measured number average molecular weight of the PDMAAm (GPC), number average molecular weight of the PDMS-Ac (NMR), weight of PDMS-Ac, and the weight of the PDMAAm produced according to the following formula:

$${M}_{c} = \frac{{M}_{n , PDMAAm ( GPC )} \times {M}_{n , PDMA \text{-} Ac ( NMR )} \times {Wt}_{PDMAAm}}{\begin{matrix}{Wt}_{PDMS \text{-} Ac} \times {M}_{n , PDMAAm ( GPC )} + \\ {M}_{n , PDMA \text{-} Ac ( NMR )} \times {Wt}_{PDMAAm}\end{matrix}} \qquad Eq . 8$$

M_{c} was also be determined from the calculated number average molecular weight of the PDMAAm, the measured number average molecular weight of the PDMS-Ac (NMR), assuming an initiator efficiency (f) of 1.0 or 0.5, weight of PDMS-Ac, and the weight of the PDMAAm produced according to the following formula:

$${M}_{c} = \frac{{M}_{n , PDMAAm ( calculated )} \times {M}_{n , PDMA \text{-} Ac ( NMR )} \times {Wt}_{PDMAAm}}{\begin{matrix}{Wt}_{PDMS \text{-} Ac} \times {M}_{n , PDMAAm ( calculated )} + \\ {M}_{n , PDMA \text{-} Ac ( NMR )} \times {Wt}_{PDMAAm}\end{matrix}} \\ Assuming f is 1.0 \text{:} \\ {M}_{c} = \frac{55 \text{,} 781 g \text{/} mol \times 11 \text{,} 832 g \text{/} mol \times 3.57 g}{3 g \times 55 \text{,} 781 g \text{/} mol + 11 \text{,} 832 g \text{/} mol \times 3.57 g} \\ {M}_{c} = 11156 g \text{/} mol \\ Assuming f is 0.5 \text{:} \\ {M}_{c} = \frac{55 \text{,} 781 g \text{/} mol / 0.5 \times 11 \text{,} 832 g \text{/} mol \times 3.57 g}{3 g \times 55 \text{,} 781 g \text{/} mol / 0.5 + 11 \text{,} 832 g \text{/} mol \times 3.57 g \times 0.5} \\ {M}_{c} = 12 \text{,} 507 g \text{/} mol \qquad Eq . 9$$

In these calculations, the calculated Mₙ of PDMAAm (here 55781 g/mol) can be used. Where f equals either 1 or 0.5, M_{c} was found to be 11156 g/mol and 12507 g/mol, respectively. The difference between these is insignificant. The minimum efficiency for AIBN at 70° C. is reported as 0.76 (M_{c}=11807).

### Example 3

### Synthesis of Poly(N,N-dimethylacrylamide)/Polydimethylsiloxane Conetworks

### a) Synthesis of 2-propionic acid 3-(1,1,3,3-tetramethyldisiloxanyl) propyl ester (SiHMA)

The synthesis strategy for SiHMA is given by the following scheme:

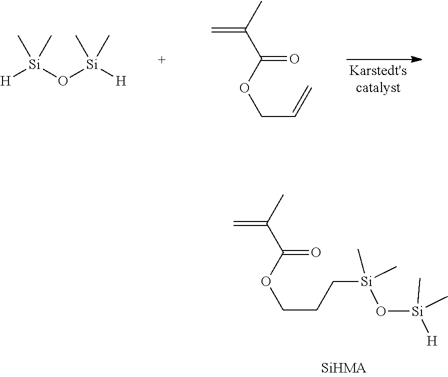

Thus, tetramethyldisiloxane (134 g, 1 mol) and allyl methacrylate (126 g, 1 mol) were placed in a round bottom flask. The reaction was started by the addition of Karstedt's catalyst (0.5 mL) and the mixture was strirred for 3 h. Then triphenylphosphine (10 mL) was added and the charge was vacuum distilled at 50° C. The product (SiHMA) is a colorless liquid with a boiling point of 62° C. Proton NMR spectroscopy confirmed tyhe expected structure.

The spectrum shows a multiplet at 4.67 ppm, which indicates the presence of the SiH group, and the characteristic resonances at 6.2 and 5.6 ppm (for the olefinic protons) and at 1.9 ppm (for the methyl protons) are associated with the methacrylate (MA) group.

### b) Synthesis of the Asymmetric-Telechelic Macromonomer (MA-PDMS-V)

Molecularly-bimodal crosslinkable branches (MA-PDMS-V) of bAPG were prepared by combining SiHMA with two different molecular weight (17,200 and 117,500 g/mol) vinyl ditelechelic PDMSs (V-PDMS-V)s by hydrosilation.

The following scheme shows the tranformations and the structure of the products:

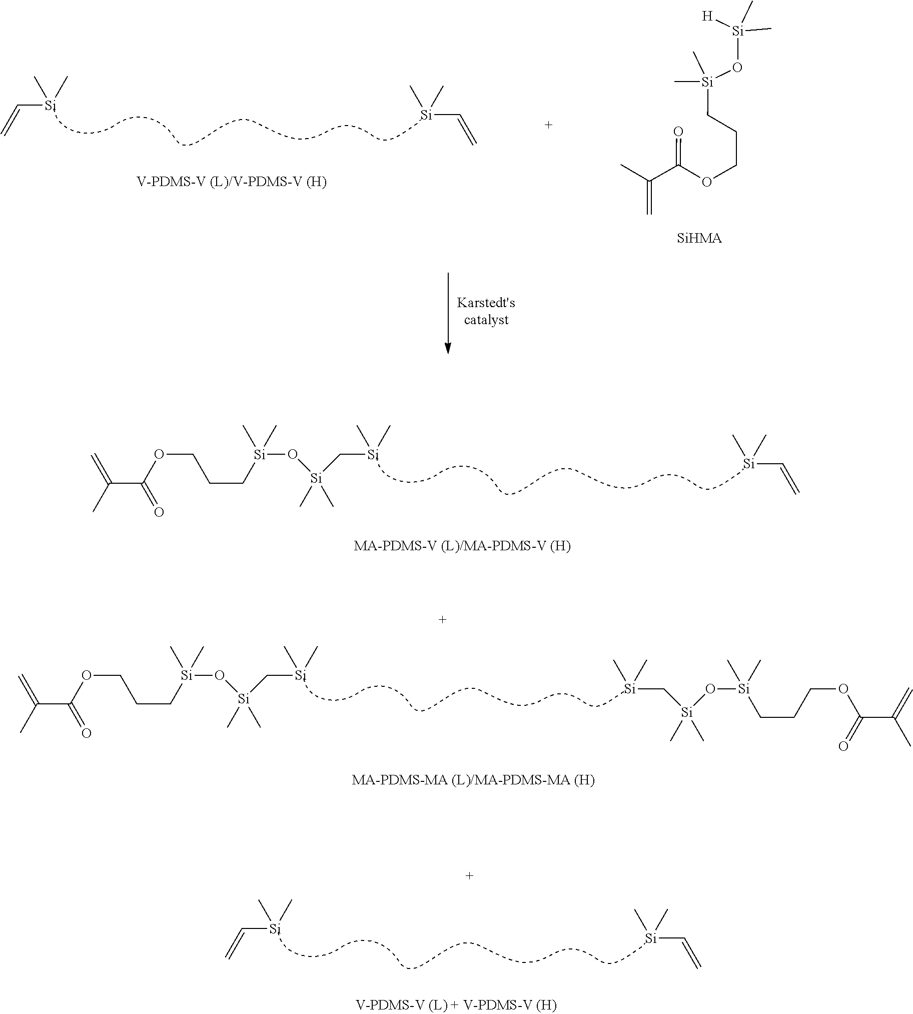

In this scheme the dotted line stands for the low or high molecular weight PDMSs.

Thus, V-PDMS-V and SiHMA were placed in a 500 mL round bottom flask and dissolved in freshly distilled toluene at room temperature. Then various compositions (1-5%) high molecular weight V-PDMS-V(H) and low molecular weigth V-PDMS-V(L) were added to the system. Reagent quantities and stoichiometry are shown in Table 1. Hydrosilation was started by the addition of Karstedt's catalysts, and the charge was stirred while heating at 50° C. for 2 h.

TABLE 1
Reaction Conditions for the Preparation of
Assymeric Telechelic MA-PDMS-V Macromer*

| | MA-PDMS-V-0 | MA-PDMS-V-1 | MA-PDMS-V-2 | MA-PDMS-V-5 |
|---|---|---|---|---|
| V-PDMS-V(L) | 0.250 mmol | 0.248 mmol | 0.245 mmol | 0.238 mmol |
| (17200 g/mol) | (100%) | (99%) | (98%) | (95%) |
| V-PDMS-V(H) | — | 2.5 × 10⁻³ mmol (1%) | 5.0 × 10⁻³ mmol | 12.5 × 10⁻³ mmol |
| (117500 g/mol) | | | (2%) | (5%) |

*Each compositions contained SiHMA = 0.25 mmol, Karstedt catalyst (3% xylene solution) = 0.02 mL, and toluene = 23 mL.

The product was characterized by ¹H NMR spectroscopy and GPC and the resonances associated with the SiH proton (4.67 ppm) disappeared. The resonance for the CH₂ protons, which arose by hydrosilation of —Si—CH═CH₂ by SiHMA, appears at 0.4 ppm.

According to the symmetrical monomodal GPC traces, the high molecular weight MA-PDMS-V was homogeneoulsy integrated into the the graft. The shift of the elution peaks toward increased molecular weights with increasing amount of MA-PDMS-V further indicates the incorporation of the high molecular weigth MA-PDMS-V.

### c) Synthesis of [PDMAAm(PDMS)]-g-PDMS-V (bAPG)

The free radical terpolymerization of DMAAm plus MA-PDMS-V and MA-PDMS-MA yields a bAPG consisting a PDMAAm backbone carrying -PDMS-V branches. The vinylsilyl termini do not copolymerize with the MA groups under free radical conditions; therefore the bAPG remains soluble. The following scheme helps to visualize the synthetic strategy:

Mixture of MA-PDMS-V(L)/MA-PDMS-V (H), MA-PDMS-MA(L)/MA-PDMS-MA (H)and V-PDMS-V(L)/V-PDMS-V (H) (see scheme in b) above):

Thus, freshly distilled DMAAm (3.57 g) , various mixtures of 0, 1, 2 and 5% low and high molecular weigth MA-PDMS-V (total=0.25 mmol), and 65 mL toluene were placed in a 500 mL round bottom flask and stirred under a nitrogen atmosphere. Then AIBN (5.36 mg) was added and the solution was stirred at 65° C. for 24 h. The solvent was evaporated under vacuum and the solid bAPG was recovered. Conversion was found to be quantitative.

Depending on the overall composition, i.e., on the amount of low and high molecular weigth MA-PDSM-V, the products were optically clear rigid (MA-PDMS-V-0 and MA-PDMS-V-1) or flexible (MA-PDMS-V-2 and MA-PDMS-V-5) materials.

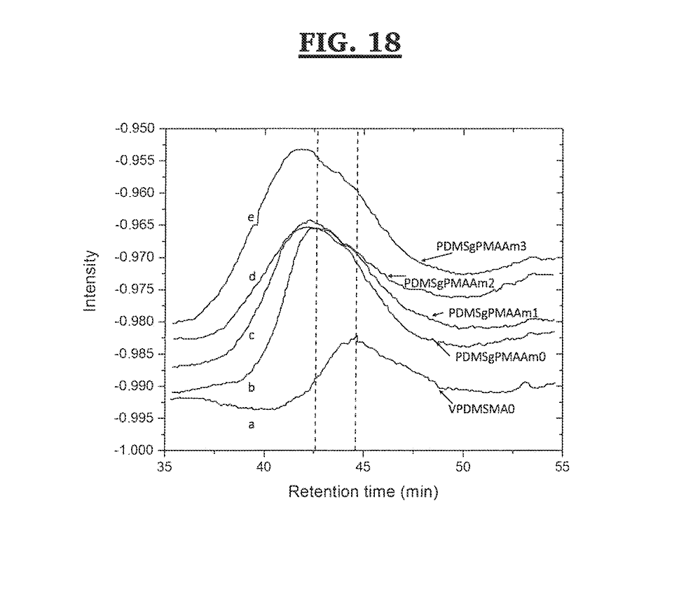

FIG. 18 shows GPC traces of the four representative grafts containing 0, 1, 2, and 5% V-PDMS-MA(H), and the V-PDMS-MA for comparison.

The position of the main elution peak of V-PDMS-MA shifts to lower retention times (higher molecular weights) with increasing V-PDMS-MA concentration, which indicates successful grafting. Moreover, the noticeable broadening of the peaks with increasing V-PDMS-MA(H) content suggests that the presence of V-PDMS-MA(H) did not affect grafting efficiency or architectural homogeneity.

### d) Crosslinking the bAPG to β-APCN and the Preparation of Membranes

The molecularly-bimodal amphiphilic graft was crosslinked by hydrosilation of the pendant -PDMS-V branches by the use of a polyhydrosiloxane-PDMS copolymer (PHMS-co-PDMS). The structure of the crosslinker was:

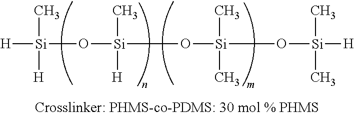

The following equation shows the network formation effected by the use of this crossliker, and the structure of the target β-APCN:

[PDMAAm (PDMS)]-g-PDMS-V

In light of the foregoing, it should be appreciated that the present invention significantly advances the art by providing a drug delivery device that is structurally and functionally improved in a number of ways. While particular embodiments of the invention have been disclosed in detail herein, it should be appreciated that the invention is not limited thereto or thereby inasmuch as variations on the invention herein will be readily appreciated by those of ordinary skill in the art. The scope of the invention shall be appreciated from the claims that follow.

1. A three layer bimodal amphiphilic co-network (β-APCN) based drug delivery device comprising:
a middle β-APCN layer comprising a drug to be administered to a patient, said middle β-APCN layer having a first drug coefficient;
a first and second outer β-APCN layer comprising a diffusional barrier material; each outer β-APCN layer having a first surface in contact with said middle β-APCN layer and a second surface in contact with a bodily fluid of the patient into whom the drug is to be delivered, wherein said first and second outer β-APCN layers have a second and third drug coefficient;
wherein said first drug coefficient is larger than said second drug coefficient and the rate of drug release from said two outer β-APCN layers into the bodily fluid of the patient is substantially independent of the concentration of said drug in said middle β-APCN layer.

2. The three layer β-APCN based drug delivery device of claim 1, wherein said middle β-APCN layer and said two outer β-APCN layers further comprise a co-network of poly(N,N-dimethylacrylamide) (PDMAAm) and polydimethylsiloxane (PDMS), crosslinked to form a β-APCN.

3. The three layer β-APCN based drug delivery device of claim 1, wherein said drug to be administered to a patient is hydrophilic.

4. The three layer β-APCN based drug delivery device of claim 1, wherein said drug to be administered to a patient is selected from the group consisting of antibiotics, antimicrobials, antifungals, pain medications, steroids, moxifloxacin hydrochloride, dexamethasone, levofloxacin, chlorhexidine, lidocaine, bupivacaine, tetracaine, cyclosporine A, timolol, dexamethasone 21-disodium phosphate, fluconazole, ofloxacin, and combinations thereof.

5. (canceled)

6. The three layer β-APCN based drug delivery device of claim 1, wherein the ration of said first drug coefficient to said second and/or third drug coefficient is greater than 1:1, but not more than about 20:1.

7. The three layer β-APCN based drug delivery device of claim 1, wherein said diffusional barrier material is selected from the group consisting of vitamin-E, nanoclays, nanoparticles, and combinations thereof.

8. The three layer β-APCN based drug delivery device of claim 1 having a hydrophilic pore size of from about 30 nm to about 50 nm.

9. The three layer β-APCN based drug delivery device of claim 1, wherein said first outer β-APCN layer is substantially impermeable to said drug and need not have a second surface in contact with the bodily fluid of the patient; and substantially all of the drug is released through said second outer β-APCN layer.

10. The three layer β-APCN based drug delivery device of claim 1 comprising one of a therapeutic contact lens and a wound dressing.

11. (canceled)

12. A method of making the three layer β-APCN based drug delivery device of claim 1 comprising:
A. preparing the middle β-APCN layer and allowing it to dry;
B. preparing a biocompatible solution comprising a drug to be delivered to a patient;
C. loading the drug into said middle β-APCN layer by placing it into said biocompatible solution comprising a drug, whereby said drug is absorbed into said middle β-APCN layer;
D. preparing a first outer β-APCN layer comprising a diffusional barrier material and a second outer β-APCN layer comprising a barrier material; said diffusional barrier material slowing the rate of diffusion of said drug through said first and second outer β-APCN layers;
E. placing said middle β-APCN layer between said first and second outer β-APCN layers; and
F. joining said first outer β-APCN layer, said middle β-APCN layer and said second outer β-APCN layer together to form the three layer β-APCN based drug delivery device of claim 1.

13. The method of claim 12 wherein said middle β-APCN layer and said first and second outer β-APCN layers comprise a co-network of poly(n, n dimethylacrylamide) (PDMAAm) and polydimethylsiloxane (PDMS), crosslinked to form a β-APCN.

14. The method of claim 12 wherein said drug to be delivered to the patient is selected from the group consisting of antibiotics, antimicrobials, antifungals, pain medications, steroids, moxifloxacin hydrochloride, dexamethasone, levofloxacin, chlorhexidine, lidocaine, bupivacaine, tetracaine, cyclosporine A, timolol, dexamethasone 21-disodium phosphate, fluconazole, ofloxacin, and combinations thereof.

15. (canceled)

16. The method of claim 12 wherein said barrier materials is selected from the group consisting of vitamin-E, nanoclays, nanoparticles, and combinations thereof.

17. The method of claim 12 wherein the step of preparing said first and second outer β-APCN layers comprises adding said diffusional barrier material during formation of the β-APCN.

18. A method of providing zero order drug release to a patient using the three layer β-APCN based drug delivery device of claim 1 comprising:
A. preparing a three layer β-APCN based drug delivery device comprising:
a middle β-APCN layer comprising a drug to be administered to a patient, said middle β-APCN layer having a first drug diffusion coefficient;
a first and second outer β-APCN layer comprising a diffusion barrier material; each outer β-APCN layer having a first surface in contact with said middle β-APCN layer and a second surface, wherein said first and second outer β-APCN layers have a second and third drug coefficient;
wherein said first drug coefficient is larger than said second and/or third drug diffusion coefficient; and
B. placing said three layer β-APCN based drug delivery device into the bodily fluid of the patient into which the drug is to be delivered so that the second surfaces of said two outer β-APCN layers are in contact with the bodily fluid of the patient into which the drug is to be delivered, wherein said drug diffuses out of the second surfaces of said first and second outer β-APCN layers and into the bodily fluid of the patient at a rate that is substantially independent of the concentration of said drug loaded into said middle β-APCN layer.

19. The method of claim 18 wherein the ratio of said first drug coefficient to said second and/or third drug coefficient is greater than 1:1 but not greater than about 20:1.

20. The method of claim 18 wherein said bodily fluid of the patient comprises, tears, blood, serum, interstitial fluid, spinal fluid, sweat, saliva, and combinations thereof.

21. The method of claim 18 wherein said three layer β-APCN based drug delivery device is a therapeutic contact lens and wherein the step of placing said three layer β-APCN based drug delivery device into the bodily fluid of the patient comprises placing said three layer β-APCN based drug delivery device between eyelid and cornea of the patient.

22. (canceled)

23. The method of claim 18 wherein said first outer β-APCN layer is substantially impermeable to said drug and need not have a second surface in contact with the bodily fluid of the patient; and substantially all of the drug is released through said second outer β-APCN layer.

24. The method of claim 23 wherein said three layer β-APCN based drug delivery device is a wound dressing and wherein the step of placing said three layer β-APCN based drug delivery device into the bodily fluid of the patient comprises placing said three layer β-APCN based drug delivery device over a wound such that said second surface of said second outer β-APCN layer is in contact with the wound.

25. (canceled)
